(19) United States
(12) Patent Application Publication
(10) Pub. No.: US 2011/0151091 A1
(43) Pub. Date: Jun. 23, 2011

(54) GLUTEN QUALITY WHEAT VARIETIES AND METHODS OF USE

(75) Inventors: Charles Ntiamoah, Chandler, AZ (US); Theresa Cogswell, Olathe, KS (US)

(73) Assignees: DOW AGROSCIENCES LLC, Indianapolis, IN (US); WORLD WIDE WHEAT, L.L.C., Tempe, AZ (US)

(21) Appl. No.: 12/953,958

(22) Filed: Nov. 24, 2010

Related U.S. Application Data
(60) Provisional application No. 61/264,599, filed on Nov. 25, 2009.
(60) Provisional application No. 61/405,124, filed on Oct. 20, 2010.
(60) Provisional application No. 61/369,566, filed on Jul. 30, 2010.
(60) Provisional application No. 61/405,071, filed on Oct. 20, 2010.

(51) Int. Cl. A21D 13/00; A23L 1/10; C12N 5/04; A01H 1/02; A21D 10/00; A01H 1/00; A01H 5/00

(52) U.S. Cl. 426/549; 426/622; 435/410; 800/260; 800/278; 800/279; 800/320.3

(57) ABSTRACT

This invention relates generally to wheat varieties comprising high gluten strength, content or quality; to identity-preserved grain products (e.g. flour) produced therefrom; and to baked goods prepared from said identity-preserved grain products. In particular examples, a wheat variety comprising high gluten strength content or quality is designated AZCABR4421W, ARGIMI7232W, COI565W, AUBR31064W, AUBR31282W, CHBR1481W, and ARGBR5945W. In some embodiments, identity-preserved grain products derived from wheat varieties comprising high gluten strength, content or quality may have a lower cost of manufacturing than other high gluten grain products. In some embodiments, identity-preserved grain products derived from wheat varieties comprising high gluten strength, content or quality may convey one or more desirable characteristics of high gluten strength, content or quality to a baked good prepared from said identity-preserved grain products; for example, greater dough elasticity, improved shape, and/or low caloric content.

### CROSS-REFERENCE TO RELATED APPLICATION

This application is a utility conversion of U.S. Provisional Patent Application Ser. No. 61/264,599, filed Nov. 25, 2009, for “White Wheat Varieties, and Compositions and Methods of Using the Same,” U.S. Provisional Patent Application Ser. No. 61/405,071, filed Oct. 20, 2010, for “White Wheat Varieties, and Compositions and Methods of Using the Same,” U.S. Provisional Patent Application Ser. No. 61/405,124, filed Oct. 20, 2010, for “Gluten Quality Wheat Varieties and Methods of Use.” and U.S. Provisional Patent Application Ser. No. 61/369,566, filed Jul. 30, 2010, for “Gluten Quality Wheat Varieties and Methods of Use.”

### FIELD OF THE DISCLOSURE

This invention relates generally to agriculture, and more particularly to cultivated wheat varieties exhibiting high gluten quality and uses thereof. In certain embodiments, the invention relates to identity-preserved milled grain products such as wheat flour manufactured from high gluten wheat varieties of the disclosure, and identity-preserved grain intermediate products such as milled bran flours manufactured from high gluten wheat varieties of the disclosure. In certain embodiments, baked goods (e.g., leavened bread) prepared from high gluten wheat varieties of the disclosure are also provided.

### BACKGROUND

Wheat is an important crop as a food staple and nutritional agent, and has been cultivated domestically for about 10,000 years. In 2007, world production of wheat was 607 million tons, which makes wheat the third most-produced cereal after maize and rice. Wheat grain is a staple food used to make flour for leavened, flat, and steamed breads, biscuits, cookies, cakes, breakfast cereal, pasta, noodles, couscous, and for fermentation to make beer, alcohol, vodka, or biofuels. Wheat is also planted to a limited extent as a forage crop for livestock and as a construction material for roofing thatch.

Wheat flour is a combination of; inter alia, starches, gluten proteins, pentosans, lipids, fiber, vitamins, and minerals. Gluten comprises of proteins, gliadin and glutenin. These proteins are conjoined with starch in the endosperm of wheat. Gliadin and glutenin comprise about 80% of the protein contained in some varieties wheat seed. These proteins are insoluble in water, and can be purified by washing away associated starch. In general, bread flours are relatively high in gluten while cake flours are low. Gluten is an important source of nutritional protein, and the gluten present in flour is essential to the preparation of leavened baked goods (e.g. bread) from the flour.

As dough develops prior to baking, gluten forms a chain-like molecular structure in an elastic network. Gluten's attainable elasticity is proportional to its content of low molecular weight glutenins, because that fraction comprises sulfur atoms responsible for the cross-linking in the network. Carbon dioxide gas formed during the leavening process is trapped within the elastic network, which causes the gas to be retained in the dough, thereby leading to expansion of the dough. The elastic gluten network also forms a matrix, within which starch granules are imbedded. Water used to make the dough is also held, to a large part, in the gluten matrix.

Plant proteins have long been classified according to their solubility, using sequential extraction in the following series of solvents: (1) water; (2) dilute salt solution; (3) aqueous alcohol; and (4) dilute acid or alkali. Osborne (1924) The vegetable proteins. London: Longmans Green and Co. Using this “Osborne classification scheme,” wheat proteins were classified as albumins, globulins, gliadins, and glutenins, respectively. However, a significant fraction of wheat proteins is excluded from the Osborne fractions because they are not extractable in all of the above-mentioned solvents. Furthermore, further research accompanied by significant improvements in tools for biochemical/genetic analysis gradually taught that the Osborne fractionation does not provide a clear separation of wheat proteins that differ, e.g., in functionality during baking. The names “gliadins” and “glutenins” are contemporarily used to indicate the functionally/biochemically related proteins, rather than the Osborne fractions. Nevertheless, the Osborne fractionation method is still extensively used in studies relating protein composition to functionality in bread-making.

From a functional point of view, two groups of wheat proteins should be distinguished: the non-gluten proteins, with either no or just a minor role in baking, and the gluten proteins, which play a major role in baking. The gluten proteins are the major storage proteins of wheat. They belong to the prolamin class of seed storage proteins. Gluten proteins are found in the endosperm of the mature wheat grain, where they form a continuous matrix around starch granules. Gluten proteins are largely insoluble in water or dilute salt solutions. Two functionally distinct group of gluten proteins can be distinguished: monomeric gliadins and polymeric (extractable and unextractable) glutenins Gliadins and glutenins are usually found in approximately equal amounts in wheat. Once the flour is moistened with water to make dough, these endosperm proteins will cooperate to form a complex throughout the mass. The elasticity of this protein complex permits the encapsulation of the carbon dioxide gas bubbles produced by the yeast or other levening agents added to the dough mixture.

Flour best suited for bread making contains proteins that form a gluten complex that will retain the shape of the bread not only during baking, but also after the bread cools. Therefore, bread bakers generally desire flour having a relatively high gluten strength to cause the bread to rise properly. On the other hand, bakers of cookies, cakes, and pastries will generally want flour having lower gluten strength, so that their products will not rise as much.

For the foregoing reasons, the development of gluten is an important determinant of the texture of baked goods. More development leads to baked goods with a relatively “chewy” texture, which is desirable in products such as pizza and bagels. Less development yields baked goods with a relatively “tender” texture. Kneading promotes the formation of gluten strands and cross-links, so the texture of a baked good depends, in part, on how extensively the dough is worked. Increased wetness of the dough also enhances gluten development. Shortening inhibits formation of cross-links, so it may be used, when a tender and flaky product (e.g. pie crust and certain pastries) is desired. Further, when gluten is an ingredient in baked goods, the baking process coagulates the gluten and contributes to stabilization of the final shape of the baked good.

In the United States, wheat is classified according to whether it is hard or soft, white or red, and winter or spring. In order to fulfill their demands, flour millers must choose between available varieties of wheats that are grown in different regions, depending upon soil and climate characteristics, and which provide different characteristic properties. For example, soft red winter wheats are typically grown in Ohio, Indiana, and areas of the Southeastern U.S. Meanwhile, soft white wheats are generally grown in the Pacific Northwest and Michigan. Hard red winter wheats are primarily grown in Kansas, Nebraska, Oklahoma, and Texas.

Hard flour, or bread flour, is relatively high in gluten, with 12% to 14% gluten content, and has elastic toughness that holds its shape well once baked. Wheat flours with relatively low gluten content are often called “soft” or “weak.” The relatively low gluten levels in soft flour results in a finer texture. Soft flour is usually divided into cake flour, which generally comprises the lowest amounts of gluten; and pastry flour, which has slightly more gluten than cake flour, but less than hard flour. Hard wheat varieties typically have higher gluten quality properties that are better suited for bread baking than soft wheat varieties. Therefore, commercial bread bakers are generally biased in favor of flours made primarily from hard wheat varieties, and these varieties are demanded by millers accordingly.

In conventional flour milling, the grain is subjected to a series of milling steps that each involves a break system comprised of a pair of break rolls and an associated set of sieves. Coarser fractions that are removed by the sieves are then subsequently milled by the following break system to progressively size-reduce the endosperm to produce flour. Traditional bulk systems of moving grain have been designed to facilitate economies of scale; they bring together small loads of grain into one large load. As such, traditional systems comingle different varieties of wheat grown in the field. Comingling of wheat varieties most often occurs in storage from the farm to the elevator, in storage to rail or barge, from the rail or barge to the elevator, or from the elevator to shipment.

Thus, most wheat flour is milled from a mixture of different wheat varieties. The proportion of each kind will typically depend upon a variety of factors, such as the amount and proportion of protein contained therein. During the milling process, the endosperm portion of the wheat kernels is separated from the bran layers through a series of breaking and screening steps. While the resulting bran is commonly relegated to breakfast cereals or animal feeds, the endosperm fraction is ground to separate flour from the coarser endosperm particles. Finally, the flour may be treated with bleaching and aging agents, enriched with vitamins, and is packaged for both domestic and commercial end-users.

While wheat varieties with advantageous characteristics theoretically could fill niche markets by being used to produce whole wheat grain products with the advantageous characteristic, different varieties of wheat are typically not kept separate through the stages between the field and the market. Therefore, the whole wheat grain products reaching consumers are typically comprised of several or more different wheat varieties. It is an aim of the present invention to provide identity-preserved high gluten wheat grain products that may be associated with the traits and characteristics of a single source high gluten wheat variety. It is further aim of the present invention to provide high gluten wheat grain products prepared only from wheat varieties exhibiting high gluten quality that may be associated with high gluten.

### SUMMARY OF THE DISCLOSURE

The following embodiments are described in conjunction with systems, tools and methods which are meant to be exemplary and illustrative, and not limiting in scope. In various embodiments, wheat varieties comprising relatively high apparent gluten content are provided. In certain embodiments, the wheat variety comprising relatively high apparent gluten content is selected from the list consisting of AZBR81207WW, AUBR6122W, CABR5437W, AZCABR4421W, ARGIMI7232W, COI565W, AUBR31064W, AUBR31282W, CHBR1481W, and ARGBR5945W. At least one wheat variety comprising relatively high apparent gluten content may be used as the source for “identity-preserved” grain products. Thus, in some embodiments, identity-preserved milled grain products comprising at least one high gluten wheat variety of the invention are provided. Also provided are methods of preparing identity-preserved milled grain products (e.g., flour) of the invention. Also provided are baked goods prepared from milled grain products of the invention. Additionally, such baked products may be unleavened or leavened and may contain a substantially uniform air cell structure with a reduced amount of added gluten in a concentration effective to make such baked product having a structure and height substantially the same as a corresponding product made with standard wheat flour, wherein the ratio of the gluten in the product is between about 0.10/lb to 0.16/lb of dough.

### BRIEF DESCRIPTION OF THE FIGURES

FIGS. 1A-1L illustrate Farinograph analysis results for wheat varieties designated AZBR81207WW, AUBR31117W, AUBR6122W, CABR5437W, AZCABR4421W, ARGIMI7232W, COI565W, AUBR31064W, AUBR31282W, CHBR1481W, and ARGBR5945W, along with KS Diamond varieties.

FIGS. 2A-2L illustrate mixing curves for wheat varieties designated AZBR81207WW, AUBR31117W, AUBR6122W, CABR5437W, AZCABR4421W, ARGIMI7232W, COI565W, AUBR31064W, AUBR31282W, CHBR1481W, and ARGBR5945W, along with KS Diamond varieties.

FIG. 3 illustrates loaf volume and internal crumb appearance for wheat varieties designated AZBR81207WW, AUBR31117W, AUBR6122W, CABR5437W, AZCABR4421W, ARGIMI7232W, COI565W, AUBR31064W, AUBR31282W, CHBR1481W, and ARGBR5945W, along with KS Diamond varieties.

### DETAILED DESCRIPTION

### I. Overview of Several Embodiments

In some embodiments, wheat varieties with high apparent gluten quality may be used, for example, to produce grain products, such as identity-preserved grain products, that themselves exhibit high gluten quality.

Typically, “high-gluten flour” or “gluten flour” that is has been treated such that starch has been removed from the grain product. Removal of starch increases the proportion of the wheat proteins in the grain product that are gluten proteins. These grain products (i.e., those typically known as “high-gluten flour” or “gluten flour”) are most commonly used as food additives to increase the elasticity of doughs to which they have been added. These grain products (i.e., those typically known as “high-gluten flour” or “gluten flour”) are rarely used to produce baked goods on their own, because, e.g., the cost of treating the grain product to remove starch makes the final grain product more expensive than its untreated counterpart. Therefore, in some embodiments, wheat varieties of the invention (which exhibit high apparent gluten quality) may be used to produce grain products (e.g., identity-preserved grain products), such as flour, that are high in gluten, but which have not been treated to remove starch (for example, after harvesting the wheat). In these and other embodiments, grain products with high gluten may be used to produce baked goods, such as, for example, leavened bread, that obtains all the benefits of high gluten, which may be obtained in some embodiments at lower cost, and with a simplified production process when compared to “high-gluten flour” or “gluten quality flour” that has been treated to remove starch.

Some exemplary benefits of high gluten in a baked good according to some embodiments of the invention (e.g. leavened bread), or in the production of a baked good according to some embodiments of the invention, may include greater dough elasticity; improved shape of the baked good; reduction of caloric content when compared to a baked good produced from one or more relatively low gluten grain product(s) with less gluten than a grain product produced using one or more high-gluten or gluten-quality wheat varieties of the invention (e.g., AZBR81207WW, AUBR6122W, CABR5437W, AZCABR4421W, ARGIMI7232W, COI565W, AUBR31064W, AUBR31282W, CHBR1481W, and ARGBR5945W); and a lower cost of manufacturing when compared to said relatively low gluten grain product(s).

### II. Terms

In order to facilitate discussion of the various embodiments of the invention, the following explanations of specific terms are provided:

Gluten strength: As used herein, the term “gluten strength” refers to both objective and subjective indicia of the gluten content of a wheat product. Gluten strength can be measured, for example, by AACC method numbers 38-10; 38-12A; and 38-20.

Gluten is an important factor in protein quality and it is formed by the interaction of storage wheat proteins (i.e., glutenin and gliadin) present in approximately equal proportions, and is also associated with lipid and pentosan during dough formation. Protein quality is based on the consideration of the potential end use rather than nutritional characteristics.

Gluten quality wheat or flour: As used herein, the term “gluten quality wheat” and “gluten quality flour” refers to wheat products that achieve improved baking volume and which allow use of less flour to achieve the same.

Tests like the Pelshenke dough ball test, the Zeleny sedimentation test, water absorption capacity of flour, and the sodium dodecyl sulfate (SDS) sedimentation volume (an estimate of the strength of the wheat or quality of gluten that depends on the degree of hydration of the proteins in the wheat, and on their degree of oxidation) can give valuable information about the baking quality of wheat. Both higher gluten content and a better gluten quality give rise to slower sedimentation and higher Zeleny test values. Pasha et al. (2007) J. Food Quality 30:438-49. The higher the SDS sedimentation volume, the higher will be the strength of the protein. The wet gluten test gives a direct indication of the amount of gluten present in flour and the oxidation status. The sedimentation value of flour depends on the wheat protein composition.

Baked goods: As used herein, the term “baked goods” refers to any food item that is cooked by convection, for example, in an oven.

Grist: As used herein, the term “grist” refers to grain that has been separated from its chaff in preparation for grinding. It can also mean grain that has been ground at a grist mill. Grist can be ground into meal or flour, depending on how coarsely it is ground.

Identity-preserved: As used herein, the term “identity-preserved” refers to grain or grain products wherein the identity of the grain, or grain from which the grain product was produced, is preserved from field to customer. The identity preservation of grains involves a system of production and delivery in which the grain is segregated based on intrinsic characteristics (such as variety or gluten content) during all stages of production, storage, and transportation. For example, an identity-preserved grain product may be segregated based on the characteristic that it comprises grain of only a single wheat variety, for example, a high gluten wheat variety. By way of additional example, an identity-preserved grain product may be segregated based on the characteristic that it comprises only grain from wheat varieties sharing a common characteristic, for example, high gluten strength, content or quality. The development of an identity-preserved grain or grain product allows for the grain or grain product to be marketed by reference to its specific attributes, rather than merely by its classification. Thus, identity-preserved grain or grain products can satisfy niche markets according to specific consumer demands for, inter alia, organic, genetically-modified, whiteness, high gluten quality, unrefined, non-genetically-engineered, and/or high amylose grain or grain products.

Grain product: As used herein, the term “grain product” refers to compositions comprising one or more constituents of one or more grains. Grain constituents include any component of a whole grain, e.g., the whole grain kernel, the germ, the bran, the endosperm, and any combination thereof. Whole grains typically refer to the germ, bran, and endosperm of a grain, and may be milled or unmilled. Refined grains typically refer to grain products in which the bran and most of or the entire germ have been removed, leaving primarily or only the endosperm. A grain product may be, for example, any combination of one or more components of a grain that have been ground into flour, cut into pieces of a variety of sizes, or used whole.

Milled grain product: Wheat milling is a mechanical method of breaking open the wheat kernel to separate as much endosperm as possible from the bran and germ, and to grind the endosperm into flour. This process substantially separates the major components of wheat from one another. As used herein, the term “milled grain product” refers to compositions comprising endosperm separated from other major components of wheat by the milling process. Refined wheat flour is produced when most of the bran and germ are separated from the endosperm.

Grain intermediate product: As used herein, the term “grain intermediate product” refers to compositions comprising wheat components other than endosperm that has been separated from the endosperm by the milling process. Bran and germ are non-limiting examples of grain intermediate products.

As used herein, the phrase “produced by recombinant genetic engineering” refers to plant varieties, e.g., wheat varieties, that have been produced using recombinant DNA technology, for example, gene deletion, and/or heterologous gene expression. These plants produced by recombinant genetic engineering are distinguished from plants produced by traditional plant breeding techniques, for example, cross-pollination, and selective breeding.

### III. Gluten Quality Wheat Varieties

Some embodiments of the invention may include gluten quality wheat varieties having high apparent gluten strength, content or quality. In particular embodiments, high gluten wheat varieties may comprise more than about 14% gluten content. Thus, high gluten wheat varieties may comprise more than about 14%; 15%; 16%; 17%; 18%; 19%; 20%; 21%; 22%; 23%; 24%; or 25% gluten content. High gluten wheat varieties of the invention may be determined by quantitatively or qualitatively measuring indicators of gluten quality known to those of skill in the art, including, for example, the mixing time needed to develop a proper gluten matrix for dough prepared from flour manufactured from grain of a particular wheat variety; “bake and shred” exhibited by a baked good prepared from dough prepared from flour manufactured from grain of a particular wheat variety (generally speaking, whole wheat leavened baked goods do not exhibit much break and shred, because of the inherent weakness of the flour, compared to leavened baked goods made from refined white flour); etc. In particular embodiments, a high gluten/gluten quality wheat variety may be AZBR81207WW, AUBR6122W, CABR5437W, AZCABR4421W, ARGIMI7232W, COI565W, AUBR31064W, AUBR31282W, CHBR1481W, and ARGBR5945W.

### IV. Identity-Preserved Grain Products

Particular embodiments of the invention include identity-preserved milled grain products. In one such embodiment, a milled grain product comprising a high gluten strength, content or quality is produced wherein grain from a particular wheat variety comprising a high gluten content (for example, more than about 14%; 15%; 16%; 17%; 18%; 19%; 20%; 21%; 22%; 23%; 24%; or 25% gluten content) has been segregated from other varieties of wheat during all stages of storage, transportation, and production (e.g., milling). In another such embodiment, a milled grain product comprising a high gluten strength, content or quality is produced wherein grain from more than one particular wheat varieties, each comprising a high gluten content (for example, more than about 14%; 15%; 16%; 17%; 18%; 19%; 20%; 21%; 22%; 23%; 24%; or 25% gluten content) has been segregated from other varieties of wheat during all stages of storage, transportation, and production (e.g., milling).

Millers typically blend different wheats to achieve the desired grain end product. In some embodiments, a gluten quality wheat variety of the invention is segregated from other varieties during milling. Thus, in each of the milling steps of inspection and storage, cleaning, and conditioning, a gluten quality wheat variety of the invention is kept separate from other wheat varieties that would otherwise contaminate the process.

Inspection and storage: Wheat typically arrives at a mill by truck, ship, barge, or rail car. Before the wheat is unloaded, samples are taken to be sure it passes inspection. X-rays may be used to detect any signs of insect infestation. Meanwhile, product control chemists may begin tests to classify the grain by milling and baking a small amount to determine end-use qualities. The results from these tests determine how the wheat will be handled and stored. The wheat will then be stored at the mill in large bins. Storing wheat is an exact science practiced by skilled artisans. The right moisture, heat, and air must be maintained, or the wheat may mildew, sprout, or ferment.

Cleaning the wheat: The first milling steps involve cleaning the wheat; equipment separates wheat from seeds and other grains, eliminates foreign materials such as metal, sticks, stones, and straw, and scours each kernel of wheat. Cleaning can take as many as, for example, six steps: (1) Magnetic Separator—the wheat first passes by a magnet that removes iron and steel particles; (2) Separator—vibrating screens remove bits of wood and straw and almost anything too big or too small to be wheat; (3) Aspirator—air currents act as a kind of vacuum to remove dust and lighter impurities; (4) De-stoner—using gravity, a machine separates the heavy material from the light material to remove stones that may be the same size as wheat kernels; (5) Disc separator—the wheat passes through a separator that identifies the size of the kernels even more closely, rejecting anything longer, shorter, more round, more angular, or in any way shaped differently than an expected kernel; and (6) Scourer—the scourer removes outer husks, crease dirt, and any smaller impurities with an intense scouring action, while currents of air pull substantially all the loosened material away.

Conditioning the wheat: The wheat is conditioned for milling through a process called “tempering.” Moisture is added in precise amounts to toughen the bran and mellow the inner endosperm. This makes the parts of the kernel separate more easily and cleanly. Tempered wheat is stored in bins from 8 to 24 hours, depending on the type of wheat (soft, medium, or hard). Blending of wheats typically is done at this time to achieve the best flour for a specific end-use.

In an impact scourer/entoleter, centrifugal force then breaks apart any unsound kernels and rejects them from the mill flow. From the entoleter, the wheat flows to grinding bins-large hoppers that will measure or feed wheat to the actual milling process. After passing through the entoleter, the wheat kernels, or berries, are in better condition than when they arrived at the mill and are ready to be milled into flour. Wheat kernels are measured or fed from the bins to the “rolls,” or corrugated rollers made from chilled cast iron. The rolls are paired and rotate inward against each other, moving at different speeds. Just one pass through the corrugated “first break” rolls begins the separation of bran, endosperm and germ. This modern milling process is a gradual reduction of wheat kernels. The goal is to produce middlings, or coarse particles of endosperm. The middlings are then graded and separated from the bran by sieves and purifiers. Each size returns to the corresponding rollers and the same process is repeated until the desired flour is obtained.

The miller's skill is demonstrated by the ability to adjust all of the rolls to the proper settings that will produce the maximum amount of high-quality flour. Grinding too hard or close results in bran powder in the flour. Grinding too open allows good endosperm to be lost in the mill's feed system. The miller must select the exact milling surface, or corrugation, on the break rolls, as well as the relation and the speed of the rollers to each other to match the type of wheat and its condition. Each break roll must be set to get as much pure endosperm as possible to the middlings rolls. The middlings rolls are set to produce as much flour as possible.

From the rolls, the grist is sent upwards to drop through sifters. The grist is moved via pneumatic systems that mix air with the particles so they flow through tubes. This is a superior method in terms of health and safety over earlier methods of moving the grist with buckets. The broken particles of wheat are introduced into rotating sifters where they are shaken through a series of bolting cloths or screens to separate the larger from the smaller particles. Inside the sifter, there may be as many as, for example, 27 frames, each covered with either a nylon or stainless steel screen, with openings that get smaller the farther they go down. Up to, for example, about six different sizes of particles may come from a single sifter, including some flour with each sifting. Larger particles are shaken off from the top, or “scalped,” leaving the finer flour to sift to the bottom. The scalped fractions are sent to other roll passages and particles of endosperm are graded by size and carried to separate purifiers.

In a purifier, a controlled flow of air lifts off bran particles while at the same time a bolting cloth separates and grades coarser fractions by size and quality.

About four or five additional break rolls, each with successively finer corrugations and each followed by a sifter, are usually used to rework the coarse stocks from the sifters and reduce the wheat particles to granular middlings that are as free from bran as possible. Germ particles will be flattened by later passage through the smooth reduction rolls and can easily be separated. The reduction rolls reduce the purified, granular middlings, or farina, to flour. The process is repeated, sifters to purifiers to reducing rolls, until the maximum amount of flour is separated, consisting of, for example, about 75% of the wheat.

There are various grades of flour produced in the milling process. Bakers buy a wide variety of flour types, based on the products they produce. The flour the consumer buys at the grocery store, called “family flour” by the milling industry, is usually a long-patent all-purpose or bread flour. Occasionally, short patent flour is available in retail stores. “Reconstituting,” or blending back together, all the parts of the wheat in the proper proportions yields whole wheat flour. This process produces higher quality whole wheat flour than is achieved by grinding the whole wheat berry. Reconstitution assures that the wheat germ oil is not spread throughout the flour so it does not readily go rancid.

The remaining percentage of the wheat kernel or berry is classified as millfeed-shorts, bran, and germ. These are examples of grain intermediate products. In some embodiments, improved wheat varieties of the invention are kept segregated from other wheat varieties during milling and all handling of the wheat prior to milling. In these embodiments, grain intermediate products obtained from the milling of that identity-preserved wheat are kept segregated from any grain intermediate products produced by milling other varieties of wheat, thereby yielding identity-preserved grain intermediate products.

Toward the end of the line in the millstream, if the flour is to be “bleached,” the finished flour flows through a device, which releases a bleaching-maturing agent in measured amounts. In the bleaching process, flour is exposed to chlorine gas or benzoyl peroxide to whiten and brighten the flour color. In some embodiments of the invention, flour produced from a variety of white wheat does not require bleaching, because the flour has a natural white color. This represents a desired result, as consumers may prefer unbleached flour with the same pleasing color characteristics as standard bleached wheat flour. The flour stream next passes through a device that measures out specified amounts of enrichment. The enrichment of flour with four B vitamins (thiamin, niacin, riboflavin) and iron began in the 1930s. In 1998, folate, or folic acid, was added to the mix of vitamin B. If the flour is self-rising, a leavening agent, salt, and calcium are also added in specified amounts.

Before the flour leaves the mill, additional lab tests are generally run to ensure that the flour conforms to the purchaser's specifications. Finally, the millstream typically flows through pneumatic tubes to the packing room or into hoppers for bulk storage. Family flour for retail sale may be packaged in, for example, from about 5 to about 25 pound bags. Bakery flour may be packaged in, for example, from about 50 to about 100 pound bags, or sent directly to bulk trucks or rail cars.

Identity-preserved grain products are produced by milling and/or processing wheat grains of a specific variety by any methods known in the art, and by additionally keeping said grains of a specific wheat variety separate from other wheat varieties at every step of the milling and/or processing.

### V. Baked Goods Produced from Identity-Preserved Grain Products

In some embodiments, identity-preserved grain products comprising high gluten wheat varieties may be used to produce baked goods, such as leavened bread, unleavened bread, bagels, crusts, pastries, cookies, crackers, and the like. In general, the practitioner may begin the baking process with a recipe or formula, and may substitute identity-preserved grain products (e.g. flour) comprising high gluten wheat varieties for standard wheat grain products according to his or her discretion in established recipes or formulas, for example, when higher gluten quality during baking is desired. In substituting identity-preserved grain products (e.g. flour) comprising high gluten wheat varieties for standard wheat grain products, the practitioner may keep in mind that identity-preserved grain products (e.g. flour) comprising high gluten wheat varieties are likely to impart higher gluten quality during baking. Therefore, some adjustment to a formula or recipe that is designed to function with standard wheat grain products may be desirable.

For example, high gluten quality generally leads to high water absorption. High absorption may lead to wet and/or gummy baked goods, unless excess water is baked out of the dough. Further, high absorption may weaken the final structure of the baked goods. Accordingly, high absorption may be balanced against other formula and process attributes by the practitioner to produce a desired result. High gluten quality also can lead to increased dough mixing time. Thus, the practitioner may adjust the baking schedule to allow for increased mixing times. Substitution of identity-preserved grain products (e.g. flour) comprising high gluten wheat varieties may also lead to a decrease in the specific volume of baked goods. However, specific volume can also be influenced by formula and/or process adjustments according to the practitioner's discretion.

Additionally, use of flour from high gluten quality sources results in less use of less vital wheat gluten to make whole wheat bread. For example, in particular embodiments, fifty percent (50%) of vital wheat gluten was required when making whole wheat bread with the high gluten quality flour compared to use of a standard, reduced gluten formula. This reduced need for additional gluten during the baking process resulted in a savings of over 16% in cost of goods to produced the resulting whole wheat bread, and further resulted in a savings of over 18% savings in the cost per loaf of bread.

### VI. Gluten Concentrations

Foods that have a structure which is based upon components of wheat flour rely, in some manner, on the action of gluten, which is a component of the wheat flour. Gluten is a mixture of proteins present in wheat and in other cereal grains. Gluten is naturally occurring in wheat flour and is advantageous in making leavened products such as bread because it has an elastic, cohesive nature which permits it to retain carbon dioxide bubbles generated by leavening agents, and therefore to form a uniform air cell structure that defines the bread.

Wheat flour has historically contained about 10% to 12% protein by weight of the flour. More recently, gluten levels in some wheat grown in the United States have dropped to a concentration that does not support acceptable air cell formation in yeast leavened dough. As a consequence, some wheat flour produced in the United States is supplemented with wheat gluten that is added to wheat flour in order to elevate the gluten to levels of about 10% to 12%. Gluten represents about 90% of the protein content of wheat flour. The protein composition of wheat gluten comprises gliadin in a concentration of about 39.1% by weight; glutenin in a concentration of about 35.1% by weight; and globulin in a concentration of about 6.75% by weight. As seen in the examples claimed all varieties have a protein level greater than 13%. This allows for reduced gluten to be added during bread making. For example, in a standard control 0.174/lb or 0.011/oz of gluten are added into dough. In higher gluten quality wheat this amount can be reduced to 0.10 to 0.16/lb or 0.004 to 0.01/oz of gluten. This can result in significant cost savings in producing a baked product or dough.

Additionally, having a quality gluten wheat allows for a reduction in the amount of protein needed for bread. This can be anywhere from a 30 to 40% reduction in the amount of protein added during the breadmaking processing. Typical 8 to 10% of bread, up to 15% is the addition of gluten. By using a higher quality gluten wheat gluten content added can be reduced from that 8-15% to as low as an addition of 8%. Thus, the amount of gluten content added can be reduced by anywhere from 1 to 8% of the total gluten added as a percentage of the entire product, preferably at least 4 to 8%. As compared to Kansas Diamond White Whole Wheat Flour which is prepared by selecting and milling hard white wheats to ensure that the process yields a light-colored, yet fiber- and protein-rich flour with microfine particles that produce a smooth, pleasing mouth feel, the amount of gluten content can be reduced by anywhere from 1 to 8% of the total gluten added as a percentage of the entire product. The Kansas Diamond White Whole Wheat Flour conforms to US Standard of Identity for whole wheat flour (21 CFR §137). As seen in the examples below, the total gluten content of a baked product may include less gluten than that as defined as conforming to US Standard of Identity for whole wheat flour (21 CFR §137).

Likewise, by means of the present invention, agronomic genes can be expressed in plants of the present invention. More particularly, plants can be genetically engineered to express various phenotypes of agronomic interest. Exemplary genes implicated in this regard include, but are not limited to, those categorized below:

1. Genes that Confer Resistance to Pests or Disease and that Encode:

A. Plant disease resistance genes. Plant defenses are often activated by specific interaction between the product of a disease resistance gene (R) in the plant and the product of a corresponding avirulence (Avr) gene in the pathogen. A plant variety can be transformed with cloned resistance genes to engineer plants that are resistant to specific pathogen strains. See, for example, Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsis RSP2 gene for resistance to Pseudomonas syringae).

B. A gene conferring resistance to a pest, such as soybean cyst nematode. See e.g., PCT Application WO 96/30517; PCT Application WO 93/19181.

C. A Bacillus thuringiensis protein, a derivative thereof or a synthetic polypeptide modeled thereon. See, for example, Geiser et al., Gene 48:109 (1986), who disclose the cloning and nucleotide sequence of a Bt δ-endotoxin gene. Moreover, DNA molecules encoding δ-endotoxin genes can be purchased from American Type Culture Collection, Manassas, Va., for example, under ATCC Accession Nos. 40098, 67136, 31995 and 31998.

D. A lectin. See, for example, the disclosure by Van Damme et al., Plant Molec. Biol. 24:25 (1994), who disclose the nucleotide sequences of several Clivia miniata mannose-binding lectin genes.

E. A vitamin-binding protein such as avidin. See PCT application US93/06487. The application teaches the use of avidin and avidin homologues as larvicides against insect pests.

F. An enzyme inhibitor, for example, a protease or proteinase inhibitor or an amylase inhibitor. See, for example, Abe et al., J. Biol. Chem. 262:16793 (1987) (nucleotide sequence of rice cysteine proteinase inhibitor); Huub et al., Plant Molec. Biol. 21:985 (1993) (nucleotide sequence of cDNA encoding tobacco proteinase inhibitor I); Sumitani et al., Biosci. Biotech. Biochem. 57:1243 (1993) (nucleotide sequence of Streptomyces nitrosporeus .alpha.-amylase inhibitor); and U.S. Pat. No. 5,494,813 (Hepher and Atkinson, issued Feb. 27, 1996).

G. An insect-specific hormone or pheromone such as an ecdysteroid or juvenile hormone, a variant thereof, a mimetic based thereon, or an antagonist or agonist thereof. See, for example, the disclosure by Hammock et al., Nature 344:458 (1990), of baculovirus expression of cloned juvenile hormone esterase, an inactivator of juvenile hormone.

H. An insect-specific peptide or neuropeptide which, upon expression, disrupts the physiology of the affected pest. For example, see the disclosures of Regan, J. Biol. Chem. 269:9 (1994) (expression cloning yields DNA coding for insect diuretic hormone receptor); and Pratt et al., Biochem. Biophys. Res. Comm. 163:1243 (1989) (an allostatin is identified in Diploptera puntata). See also U.S. Pat. No. 5,266,317 to Tomalski et al., who disclose genes encoding insect-specific, paralytic neurotoxins.

I. An insect-specific venom produced in nature by a snake, a wasp, etc. For example, see Pang et al., Gene 116:165 (1992), for disclosure of heterologous expression in plants of a gene coding for a scorpion insectotoxic peptide.

J. An enzyme responsible for a hyperaccumulation of a monoterpene, a sesquiterpene, a steroid, hydroxamic acid, a phenylpropanoid derivative or another non-protein molecule with insecticidal activity.

K. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23:691 (1993), who teach the nucleotide sequence of a cDNA encoding tobacco hornworm chitinase; and Kawalleck et al., Plant Molec. Biol. 21:673 (1993), who provide the nucleotide sequence of the parsley ubi4-2 polyubiquitin gene.

L. A molecule that stimulates signal transduction. For example, see the disclosure by Botella et al., Plant Molec. Biol. 24:757 (1994), of nucleotide sequences for mung bean calmodulin cDNA clones; and Griess et al., Plant Physiol. 104:1467 (1994), who provide the nucleotide sequence of a maize calmodulin cDNA clone.

M. A hydrophobic moment peptide. See PCT application WO 95/16776 (disclosure of peptide derivatives of Tachyplesin which inhibit fungal plant pathogens) and PCT application WO 95/18855 (teaches synthetic antimicrobial peptides that confer disease resistance).

N. A membrane permease, a channel former or a channel blocker. For example, see the disclosure of Jaynes et al., Plant Sci. 89:43 (1993), of heterologous expression of a cecropin-β, lytic peptide analog to render transgenic tobacco plants resistant to Pseudomonas solanacearum.

O. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. rev. Phytopathol. 28:451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id.

P. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland) (1994) (enzymatic inactivation in transgenic tobacco via production of single-chain antibody fragments).

Q. A virus-specific antibody. See, for example, Tavladoraki et al., Nature 366:469 (1993), who show that transgenic plants expressing recombinant antibody genes are protected from virus attack.

R. A developmental-arrestive protein produced in nature by a pathogen or a parasite. Thus, fungal endo α-1,4-D-polygalacturonases facilitate fungal colonization and plant nutrient release by solubilizing plant cell wall homo-α-1,4-D-galacturonase. See Lamb et al., Bio/Technology 10:1436 (1992). The cloning and characterization of a gene which encodes a bean endopolygalacturonase-inhibiting protein is described by Toubart et al., Plant J. 2:367 (1992).

S. A developmental-arrestive protein produced in nature by a plant. For example, Logemann et al., Bio/Technology 10:305 (1992), have shown that transgenic plants expressing the barley ribosome-inactivating gene have an increased resistance to fungal disease.

2. Genes that Confer Resistance to an Herbicide:

A. An herbicide that inhibits the growing point or meristem, such as an imidazolinone or a sulfonylurea. Exemplary genes in this category code for mutant ALS and AHAS enzyme as described, for example, by Lee et al., EMBO J. 7:1241 (1988); and Mild et al., Theon. Appl. Genet. 80:449 (1990), respectively.

B. Glyphosate (resistance conferred by, e.g., mutant 5-enolpyruvylshikimate-3-phosphate synthase (EPSPs) genes (via the introduction of recombinant nucleic acids and/or various forms of in vivo mutagenesis of native EPSPs genes), aroA genes and glyphosate acetyl transferase (GAT) genes, respectively), other phosphono compounds such as glufosinate (phosphinothricin acetyl transferase (PAT) genes from Streptomyces species, including Streptomyces hygroscopicus and Streptomyces viridichromogenes), and pyridinoxy or phenoxy proprionic acids and cyclohexones (ACCase inhibitor-encoding genes), See, for example, U.S. Pat. No. 4,940,835 to Shah, et al. and U.S. Pat. No. 6,248,876 to Barry et. al., which disclose nucleotide sequences of forms of EPSPs which can confer glyphosate resistance to a plant. A DNA molecule encoding a mutant aroA gene can be obtained under ATCC accession number 39256, and the nucleotide sequence of the mutant gene is disclosed in U.S. Pat. No. 4,769,061 to Comai. European patent application No. 0 333 033 to Kumada et al., and U.S. Pat. No. 4,975,374 to Goodman et al., disclose nucleotide sequences of glutamine synthetase genes which confer resistance to herbicides such as L-phosphinothricin. The nucleotide sequence of a PAT gene is provided in European application No. 0 242 246 to Leemans et al., DeGreef et al., Bio/Technology 7:61 (1989), describe the production of transgenic plants that express chimeric bar genes coding for PAT activity. Exemplary of genes conferring resistance to phenoxy proprionic acids and cyclohexones, such as sethoxydim and haloxyfop are the Acc1-S1, Acc1-S2 and Acc1-S3 genes described by Marshall et al., Theon. Appl. Genet. 83:435 (1992). GAT genes capable of conferring glyphosate resistance are described in WO 2005012515 to Castle et. al. Genes conferring resistance to 2,4-D, fop and pyridyloxy auxin herbicides are described in WO 2005107437 and U.S. patent application Ser. No. 11/587,893, both assigned to Dow AgroSciences LLC.

C. An herbicide that inhibits photosynthesis, such as a triazine (psbA and gs+ genes) or a benzonitrile (nitrilase gene). Przibila et al., Plant Cell 3:169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441, and 67442. Cloning and expression of DNA coding for a glutathione S-transferase is described by Hayes et al., Biochem. J. 285:173 (1992).

3. Genes that Confer or Contribute to a Value-Added Trait, Such as:

A. Modified fatty acid metabolism, for example, by transforming a plant with an antisense gene of stearyl-ACP desaturase to increase stearic acid content of the plant. See Knultzon et al., Proc. Natl. Acad. Sci. U.S.A. 89:2624 (1992).

B. Decreased phytate content-1) Introduction of a phytase-encoding gene would enhance breakdown of phytate, adding more free phosphate to the transformed plant. For example, see Van Hartingsveldt et al., Gene 127:87 (1993), for a disclosure of the nucleotide sequence of an Aspergillus niger phytase gene. 2) A gene could be introduced that reduced phytate content. In maize for example, this could be accomplished by cloning and then reintroducing DNA associated with the single allele which is responsible for maize mutants characterized by low levels of phytic acid. See Raboy et al., Maydica 35:383 (1990).

C. Modified carbohydrate composition effected, for example, by transforming plants with a gene coding for an enzyme that alters the branching pattern of starch. See Shiroza et al., J. Bacteol. 170:810 (1988) (nucleotide sequence of Streptococcus mutants fructosyltransferase gene); Steinmetz et al., Mol. Gen. Genet. 20:220 (1985) (nucleotide sequence of Bacillus subtilis levansucrase gene); Pen et al., Bio/Technology 10:292 (1992) (production of transgenic plants that express Bacillus lichenifonnis α-amylase); Elliot et al., Plant Molec. Biol. 21:515 (1993) (nucleotide sequences of tomato invertase genes); Sogaard et al., J. Biol. Chem. 268:22480 (1993) (site-directed mutagenesis of barley α-amylase gene); and Fisher et al., Plant Physiol. 102:1045 (1993) (maize endosperm starch branching enzyme II).

D. Abiotic Stress Tolerance which includes resistance to non-biological sources of stress conferred by traits such as nitrogen utilization efficiency, altered nitrogen responsiveness, drought resistance cold, and salt resistance. Genes that affect abiotic stress resistance (including but not limited to flowering, ear and seed development, enhancement of nitrogen utilization efficiency, altered nitrogen responsiveness, drought resistance or tolerance, cold resistance or tolerance, and salt resistance or tolerance) and increased yield under stress.

The examples presented herein are provided for illustrative purposes only and not to limit the scope of any embodiment of the present invention.

### EXAMPLES

Eleven gluten quality wheat breeding lines (i.e., AZBR81207WW, AUBR31117W, AUBR6122W, CABR5437W, AZCABR4421W, ARGIMI7232W, COI565W, AUBR31064W, AUBR31282W, CHBR1481W, and ARGBR5945W) and one commercially registered variety of white wheat (Kansas Diamond) were milled to produce whole grain (WG) flour. The flour was then analysed for gluten strength through a variety of tests. Baking characteristics were evaluated using a micro test baking method. The samples were designated by the following Batch numbers:

Gluten Quality Wheat Samples

| BATCH # | VARIETY |
|---|---|
| A | AZBR81207WW |
| B | AUBR31117W |
| C | AUBR6122W |
| D | CABR5437W |
| E | AZCABR4421W |
| F | KANSAS DIAMOND |
| G | ARGIMI7232W |
| H | COI565W |
| I | AUBR31064W |
| J | AUBR31282W |
| K | CHBR1481W |
| L | ARGBR5945W |

### Example 1

### Milling

Samples were tempered to 15.5% moisture content for 20 hrs and then milled on a Brabender Quadramat Jr. according to internal procedures established at CIGI. The bran fraction was then reduced using a pin mill (20,000 rpm) and added back to the milled flour to produce WG flour. The granulation of the WG flour was evaluated using an appropriate series of standard sieves on a Baler laboratory sifter strength, content, or quality.

### Example 2

### Sample Analysis

Wheat samples were analyzed for particle size index (PSI; AACC 55-30) to determine kernel hardness characteristics. Flour samples were analyzed for protein content (Williams et al., 1998), farinograph (AACC 54-21, constant flour weight procedure, 50 g bowl), moisture content (AACC 44-15A, one-stage procedure).

In order to evaluate the gluten properties of the samples, a portion of flour was sieved using a 60 wire (340 μm) screen to remove the coarser bran fraction as preliminary testing showed that this interfered with gluten analysis. This sieved fraction was then analyzed for wet gluten content and gluten index using the Glutomatic (GI; AACC 38-12A, two-stage procedure), gluten deflection time and relaxation using the Glutograph (manufacturer's instructions; 50 g weight) and gluten extensibility using the Kieffer Rig with the TA.HD Plus texture analyzer (Sopiwnyk, 1999).

### Example 3

### Baking Performance

Micro test baking performance of WG flour samples were evaluated based on the no time dough (NTD) process. Flour samples (35 g) were treated with sugar (8%), salt (2%), canola oil (3%), yeast (4%), ammonium phosphate (0.1%), ascorbic acid (60 ppm) and amylase (60 ppm). Flour was mixed to peak dough development time plus an additional 10% as determined using RAR software for capturing mixer energy input. The optimally mixed dough was rested for 10 min, scaled at 40 g, rounded, rested an additional 10 min, sheeted thru a 3/16 and a ⅛ inch gap and finally molded before being placed in the proofer (37° C./98.6° F., 85% RH). Proofing time was set as the amount of time a dummy WG dough (CWRS) took to reach 48 mm in height plus an additional 2 min due to the inherently stronger dough properties of the CWRS class of wheat. Fully proofed doughs were baked for 20 min at 375° F. Samples were cooled, and then evaluated for loaf volume by TexVol, external appearance, internal crumb color and internal texture according to established CIGI procedures.

### Example 4

### Farinograph Analysis

In baking, a Farinograph measures specific properties of flour and is used as a tool to measure shear (fluid) and viscosity of a mixture of flour and water. The primary units of the farinograph are Brabender Units (BU), an arbitrary unit of measuring the viscosity of a fluid. A baker can formulate end product by using the Farinograph's results to determine water absorption, dough viscosity (including peak water to gluten ratio prior to gluten breakdown), peak mixing time to arrive at desired water/gluten ratio, the stability of flour under mixing, and the tolerance of flour's gluten.

The farinograph is drawn on a curved graph with the vertical axis labeled in BU and the horizontal axis labeled as time in minutes. The graph is generally hockey-stick shaped, with the curve being more or less acute depending on the quality of the gluten in the flour. The points of interest on the graph include:

1. Arrival Time (Absorption)—Absorption is the point chosen by the baking industry which represents a target water to flour ratio in bread. This ratio is marked at the 500 BU line and is taken as a rule of thumb for desired taste, texture, and dough performance during proofing and baking. All other measurements are based on this 500 BU standard. Arrival time indicates the rate of absorption (minutes/BU).

2. Peak time—Peak time is reached at the highest point on the curve and indicates when the dough has reached is maximum viscosity before gluten strands begin to break down.

3. Mixing Tolerance Index (MTI)—MTI is found by taking the difference in BU between the peak time point and 5 minutes after peak time is reached. This is used by bakers to determine the amount that a dough will soften over a period of mixing. MTI may be expressed as a value in BU or as a percentage of BU lost over time

4. Departure Time—Departure time is defined as the point at which the top of the curve goes below the 500 BU line. This point is generally considered the point at which gluten is breaking down and dough has become over mixed.

5. Stability—Stability is the point between arrival time and departure time and generally indicates the quality of flour (how much gluten a flour has and how strong it is).

By way of example, a gluten rich bread flour has a stability time that is relatively long with a MTI above the 500 BU line. A weaker flour, such as a cake or pastry flour with a much lower gluten content, would have a much steeper decline after peak time.

The Farinograph is used worldwide by bakers and food technicians in building bakery formulations. The farinograph gives the baker a good snapshot of the flour's properties and how the flour will react in different stages of baking. It assists the baker in choosing the right flour for the job they are trying to complete.

These points may be used, for example, to determine the arrival time as a bare minimum time when planning full product floor time for a batch of dough. The MTI may also be used as guideline to judge the response of dough to the addition of other ingredients. Peak time may be used as a target mix time for optimal gluten structure and resilience. Stability may be used as a method of determining desired cell structure before irreparable gluten breakdown occurs.

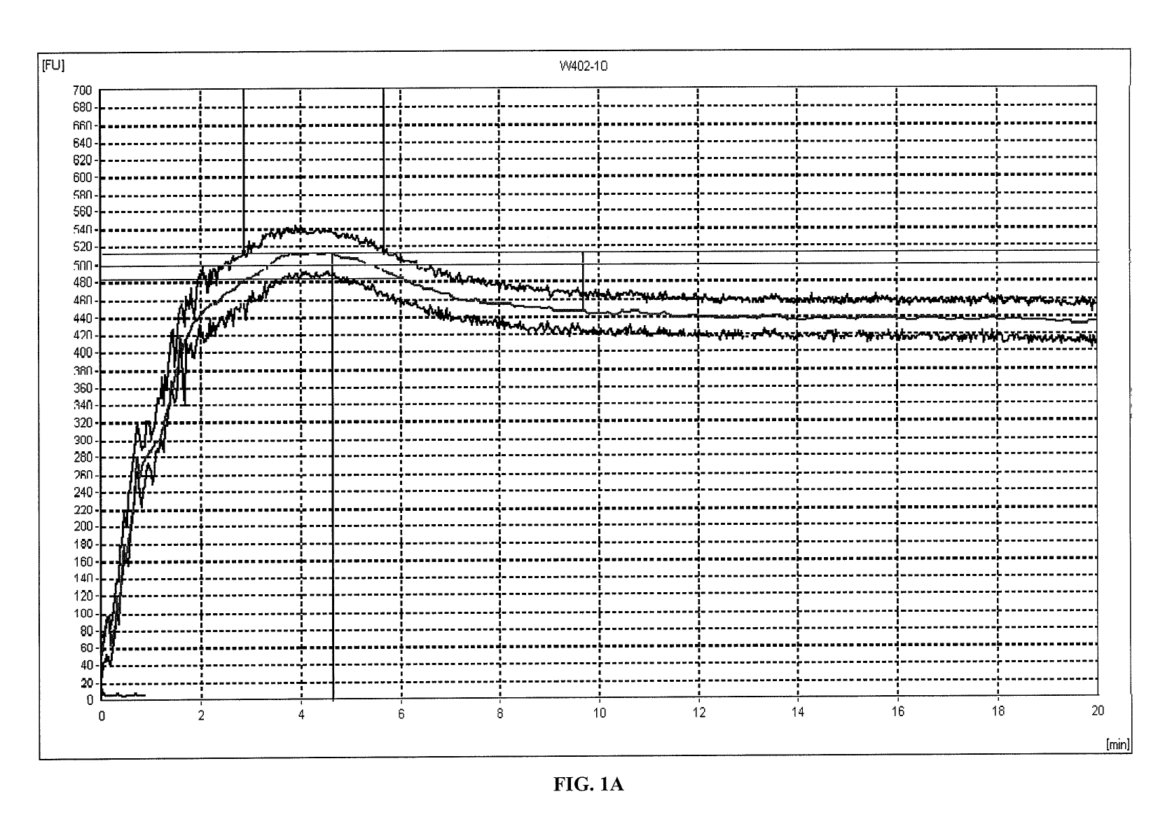

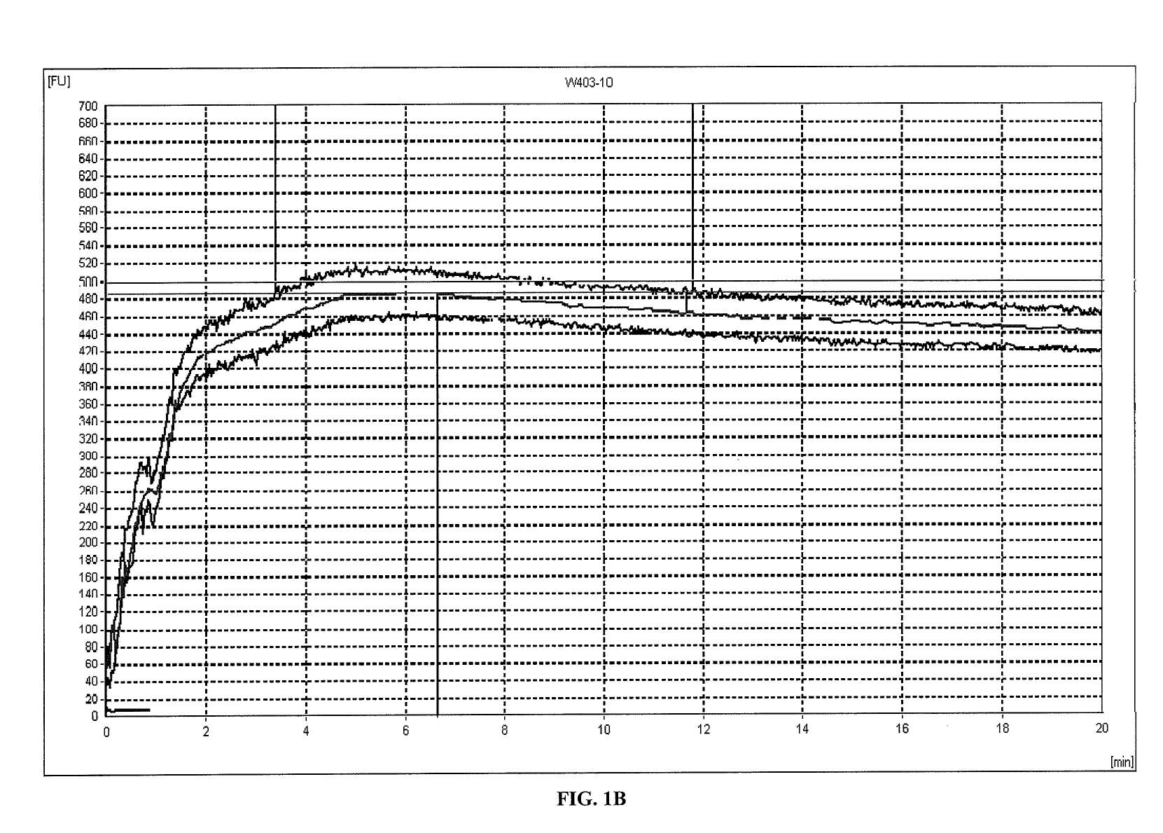

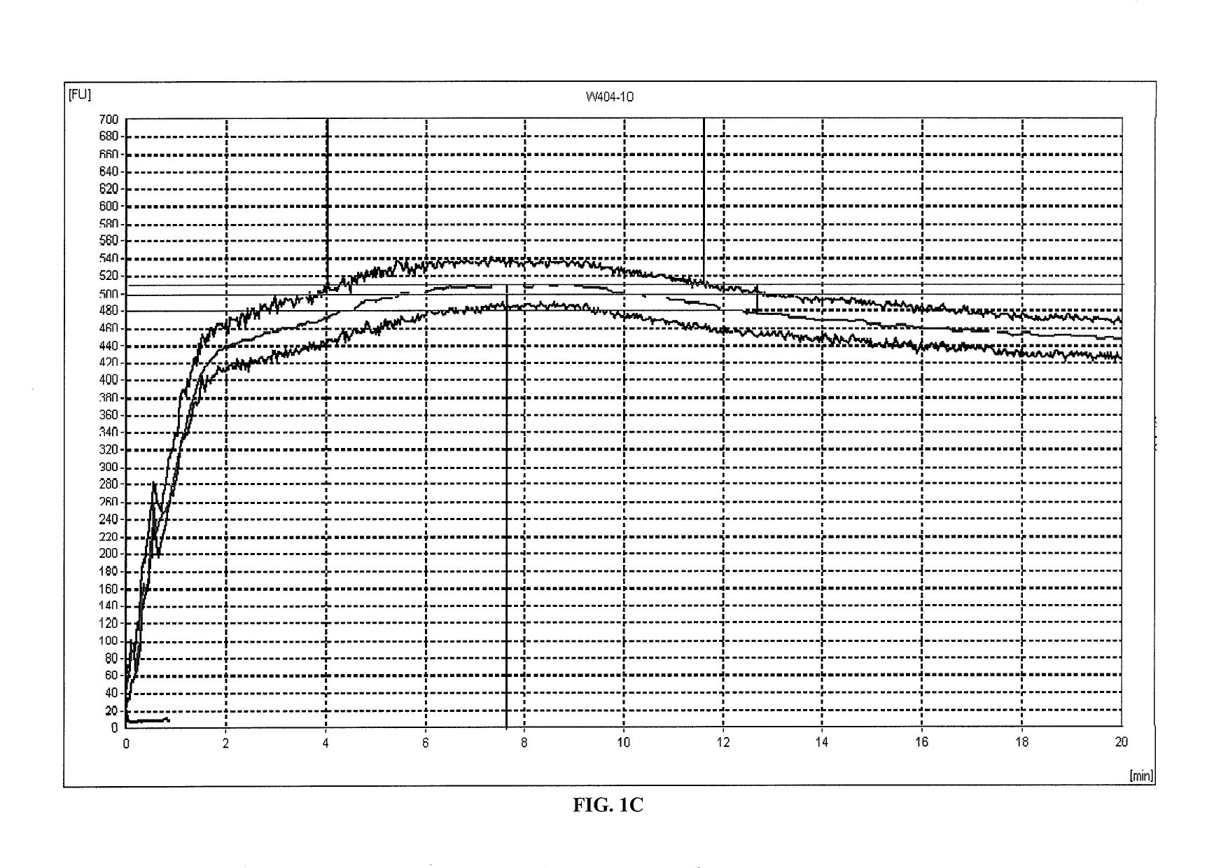

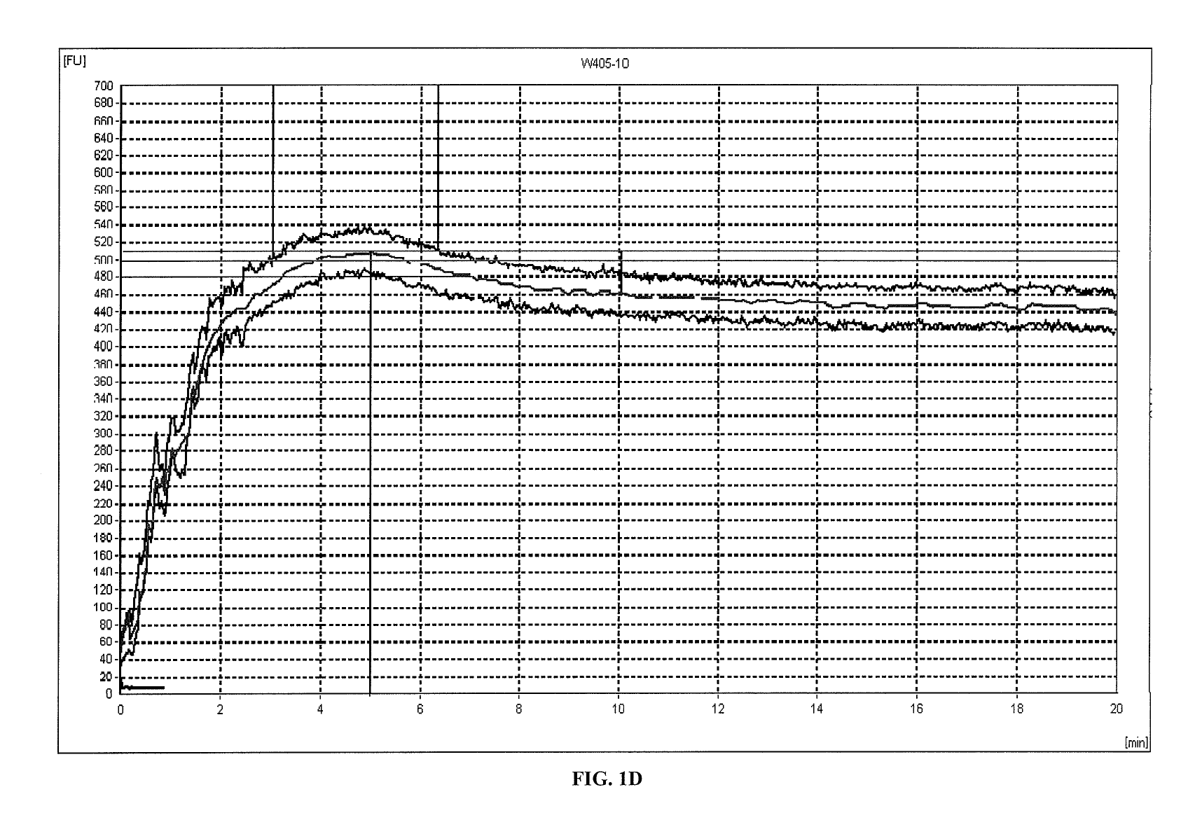

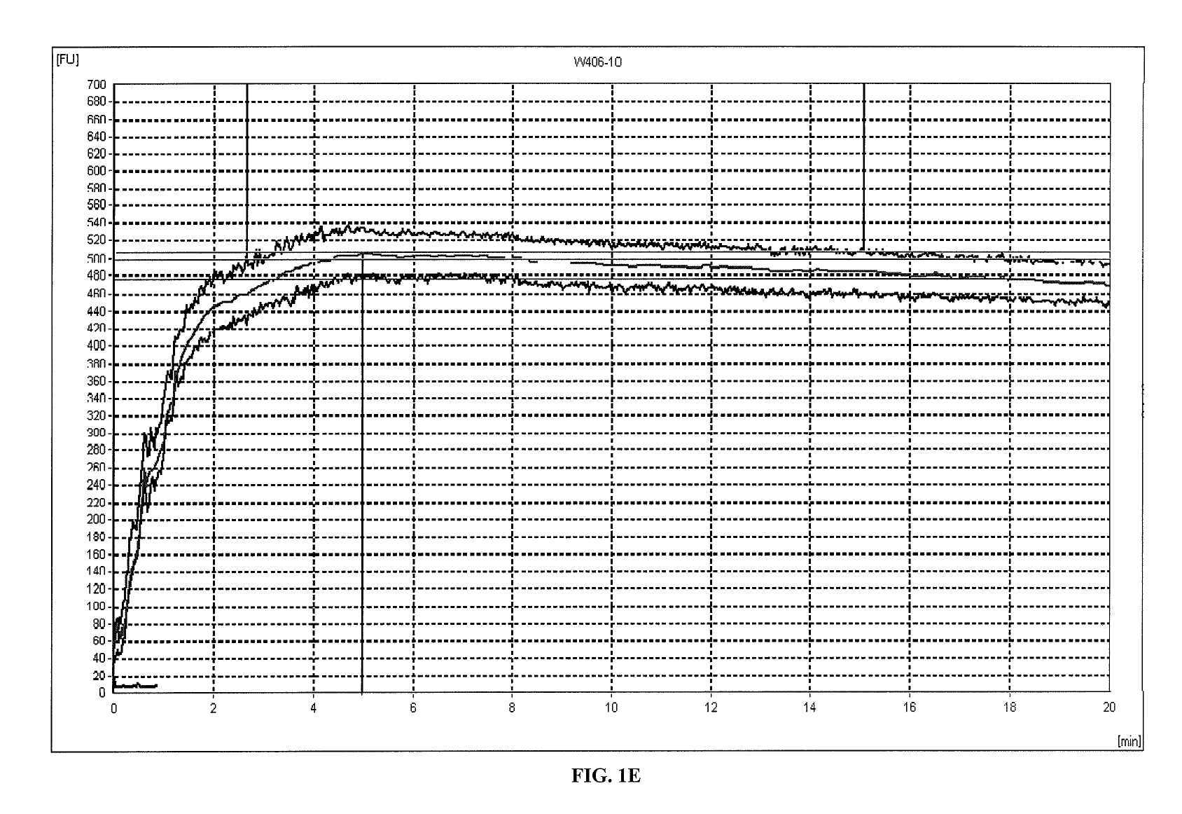

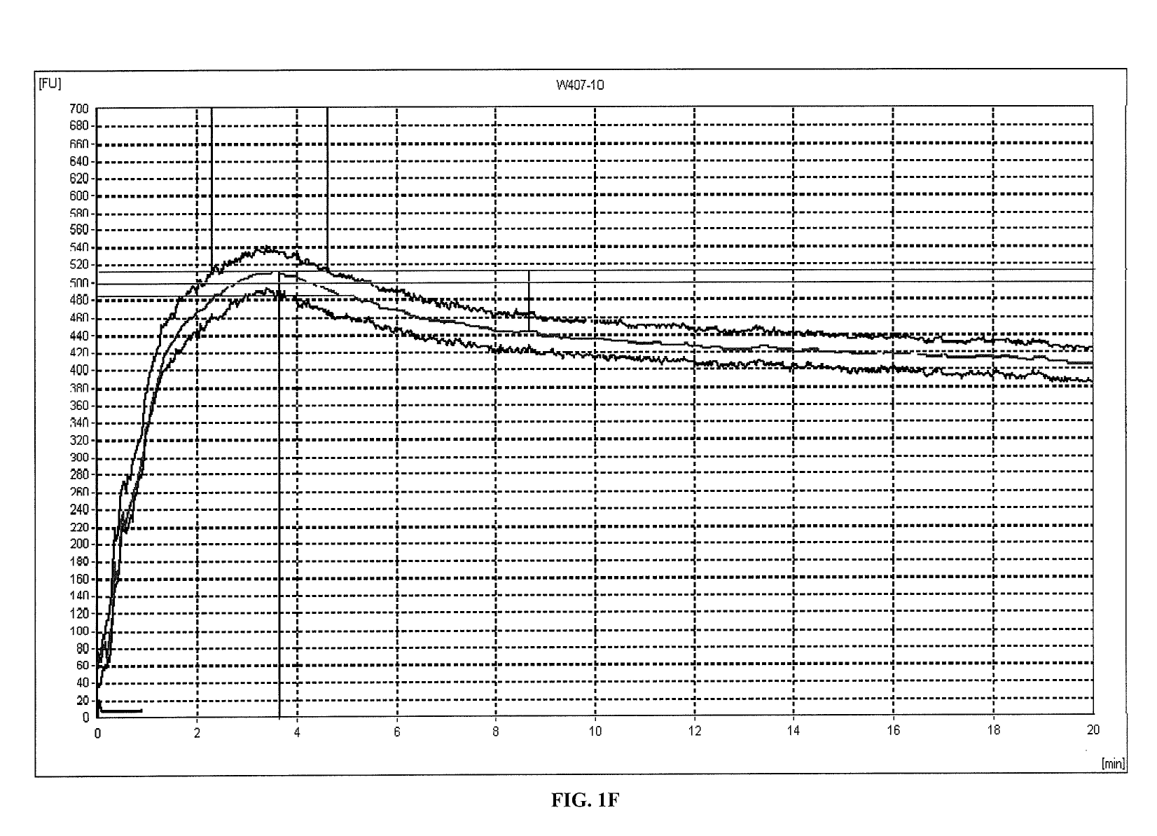

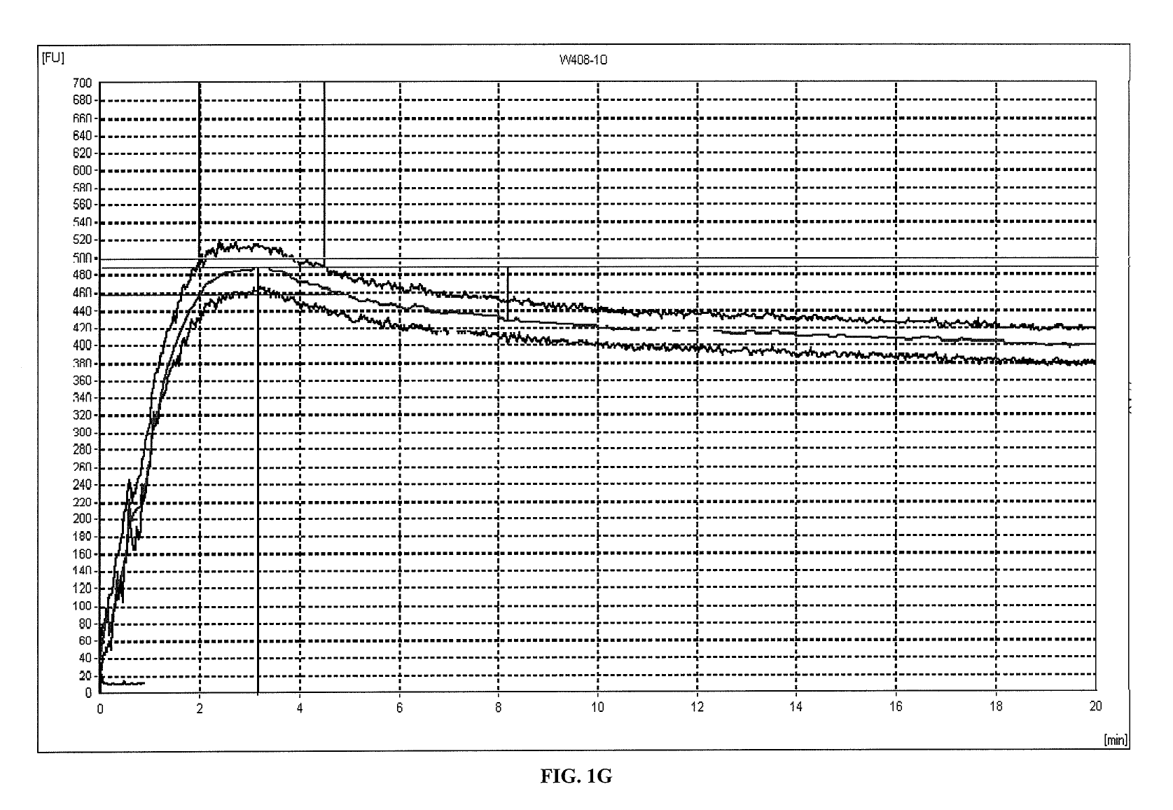

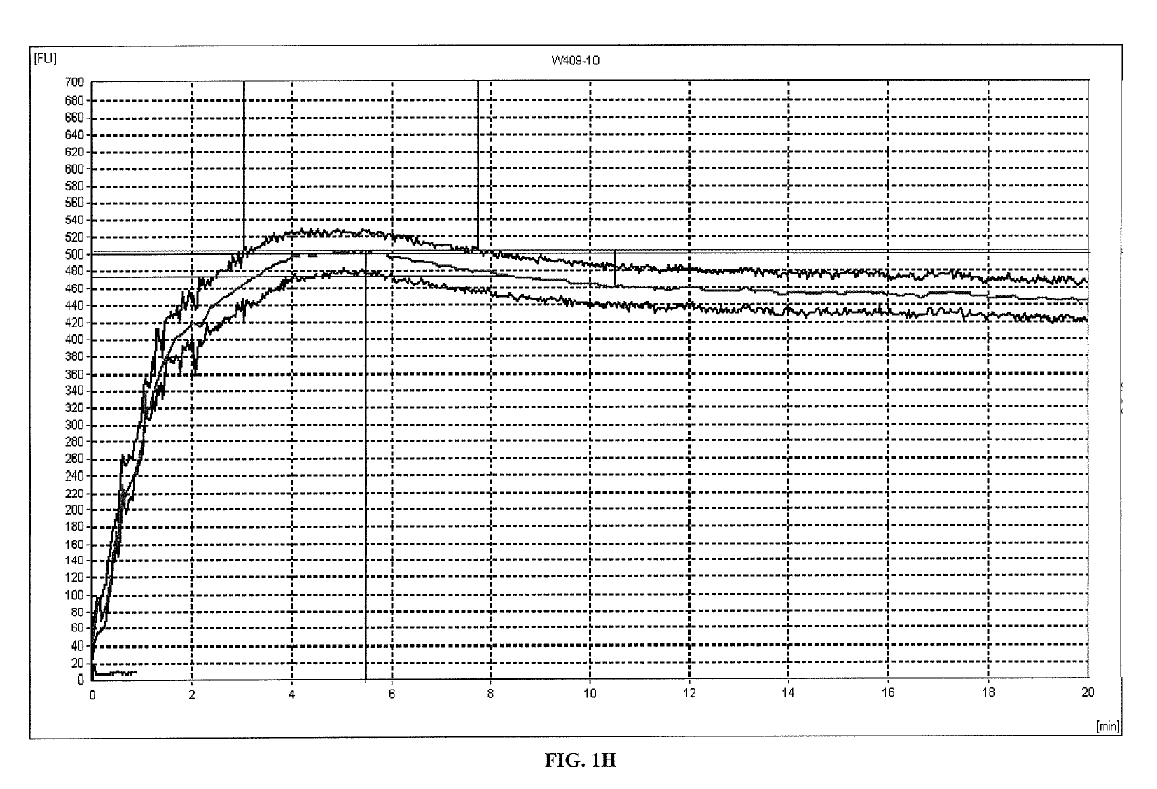

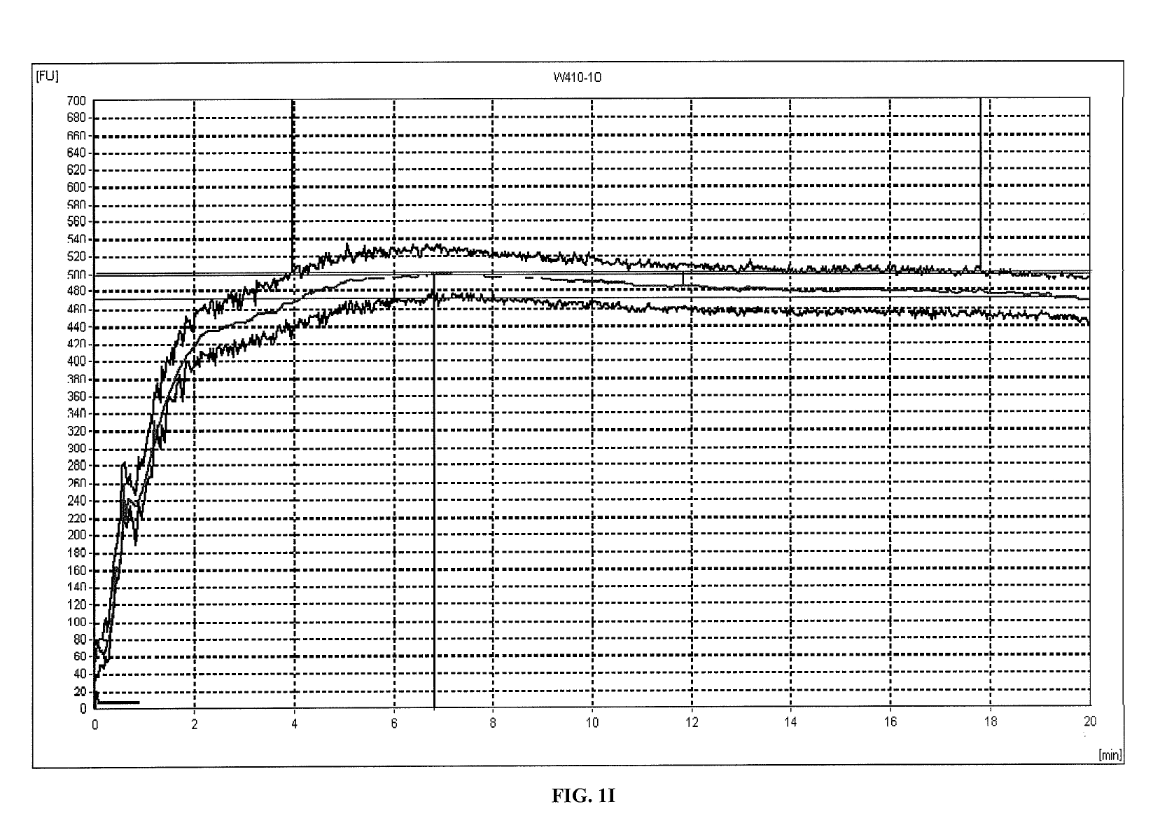

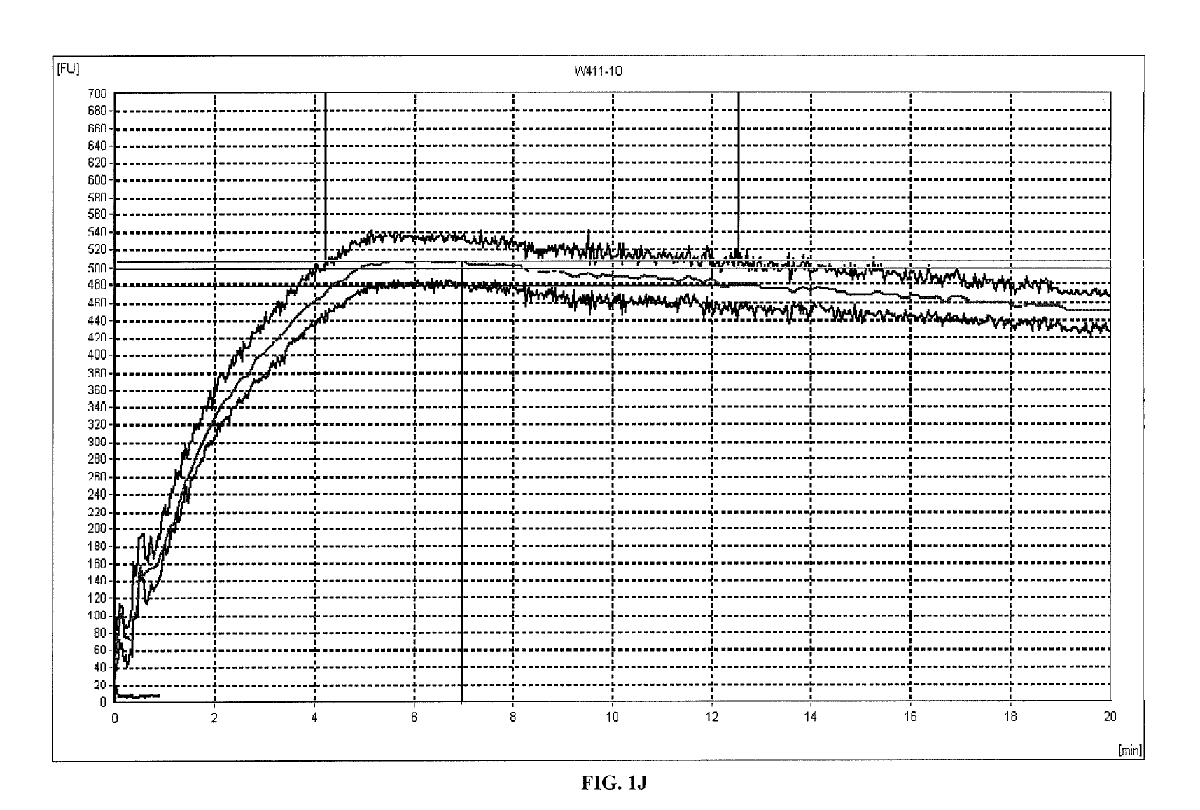

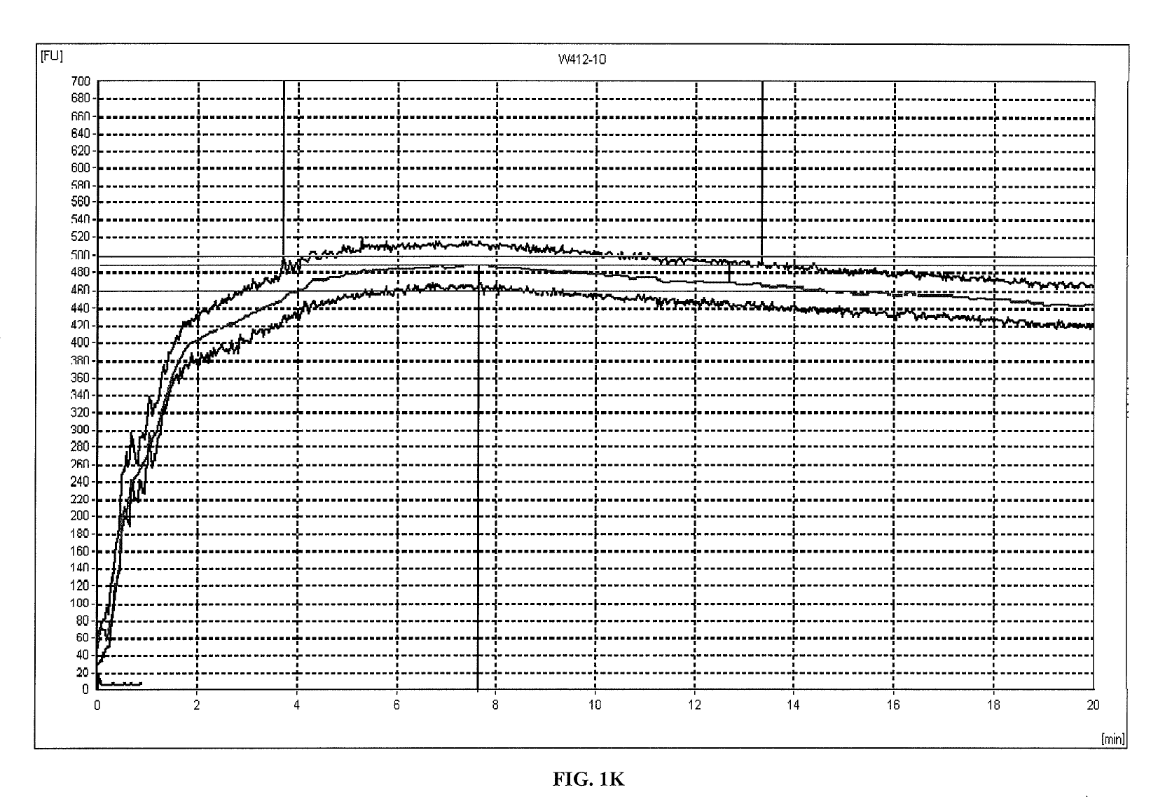

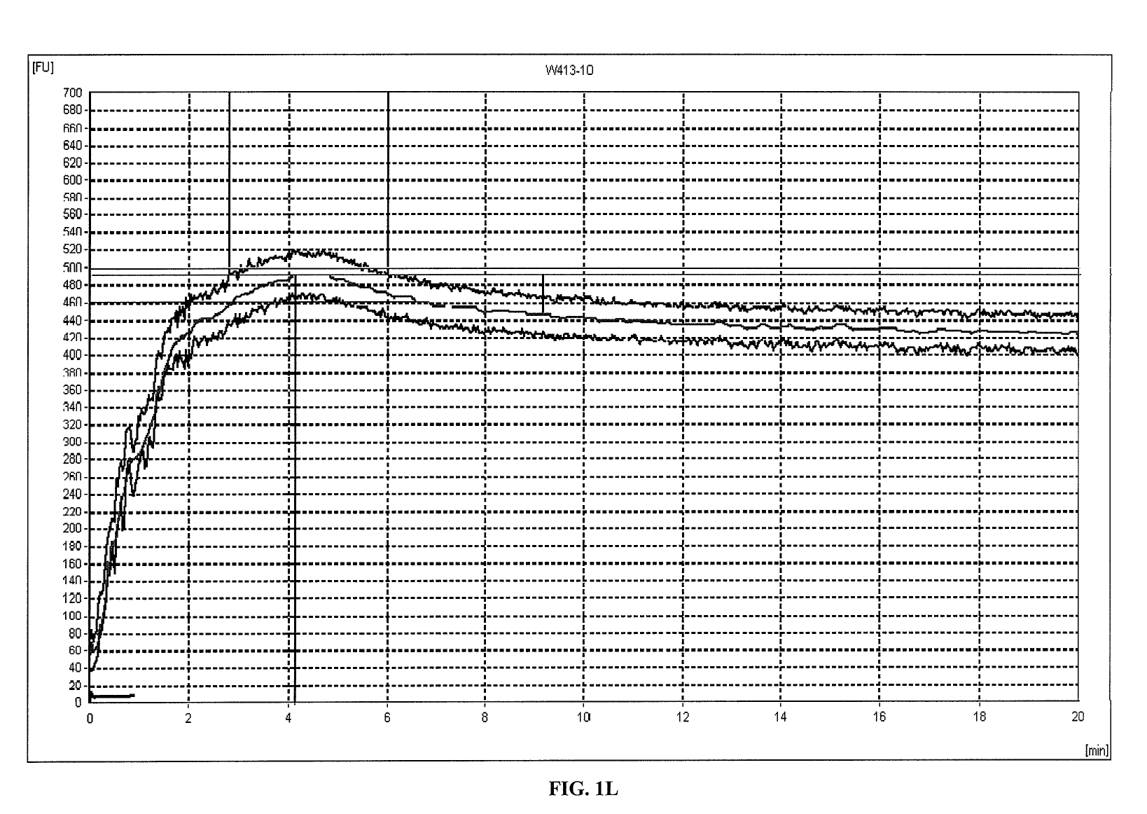

FIGS. 1A-1L show Farinograph analysis results for wheat varieties designated AZBR81207WW, AUBR31117W, AUBR6122W, CABR5437W, AZCABR4421W, ARGIMI7232W, COI565W, AUBR31064W, AUBR31282W, CHBR1481W, and ARGBR5945W, and compares the same with KS Diamond variety. The analysis and the related Farinograms indicate that wheat varieties AZBR81207WW, AUBR6122W, CABR5437W, AZCABR4421W, ARGIMI7232W, COI565W, AUBR31064W, AUBR31282W, CHBR1481W, and ARGBR5945W, are high gluten wheat varieties. Therefore, identity-preserved grain products produced from one or more of these varieties comprise high gluten wheat strength, content or quality.

### Example 5

### Results

All samples had similar granulation, with the exception of sample G which showed a slightly higher amount of product over 340 μm and a lower amount of product with granulation over 212 μm.

Analytical results are presented in Tables 1a through 1c.

TABLE 1a
Milling and Analytical Results (samples A through D)

| Sample | A | B | C | D |
|---|---|---|---|---|
| ID No. | W402-10 | W403-10 | W404-10 | W405-10 |

WHEAT (13.5% mb)

| | | | | |
|---|---|---|---|---|
| Moisture, % | 10.1 | 10.4 | 10.8 | 9.3 |
| Particle size | 37 | 47 | 46 | 40 |
| index, % | | | | |

MILLING YIELD

| | | | | |
|---|---|---|---|---|
| Flour yield | 100.0 | 100.0 | 100.0 | 100.0 |
| (Whole grain flour), | | | | |
| % | | | | |

GRANULATION

| | | | | |
|---|---|---|---|---|
| Over 60w | 11 | 11 | 12 | 11 |
| (340 μm), % | | | | |
| Over 6xx | 13 | 10 | 11 | 13 |
| (212 μm), % | | | | |
| Over 9xx | 23 | 24 | 25 | 25 |
| (150 μm), % | | | | |
| Thrus, % | 53 | 55 | 52 | 51 |
| Total recovery, % | 100 | 100 | 100 | 100 |

FLOUR (14.0% mb)

| | | | | |
|---|---|---|---|---|
| Protein content (CNA), | 15.2 | 13.3 | 13.0 | 13.0 |
| % | | | | |
| Wet gluten, % | 37.9 | 33.0 | 33.7 | 32.0 |
| Minolta color - L* | 82.8 | 84.8 | 85.3 | 84.7 |
| a* | 1.49 | 0.78 | 0.65 | 0.86 |
| b* | 12.7 | 10.5 | 11.2 | 10.9 |
| Moisture, % | 12.7 | 12.9 | 12.8 | 13.2 |

TABLE 1b
Milling and Analytical Results (samples E through H)

| | | | | |
|---|---|---|---|---|
| Sample | E | F | G | H |

WHEAT (13.5% mb)

| | | | | |
|---|---|---|---|---|
| Moisture, % | 11.3 | 10.2 | 9.8 | 9.2 |
| Particle size | 47 | 44 | 64 | 48 |
| index, % | | | | |

MILLING YIELD

| | | | | |
|---|---|---|---|---|
| Flour yield | 100.0 | 100.0 | 100.0 | 100.0 |
| (Whole grain flour), | | | | |
| % | | | | |

GRANULATION

| | | | | |
|---|---|---|---|---|
| Over 60w | 12 | 11 | 16 | 12 |
| (340 μm), % | | | | |
| Over 6xx | 10 | 12 | 5 | 12 |
| (212 μm), % | | | | |
| Over 9xx | 24 | 25 | 23 | 22 |
| (150 μm), % | | | | |
| Thrus, % | 54 | 52 | 56 | 54 |
| Total recovery, % | 100 | 100 | 100 | 100 |

FLOUR (14.0% mb)

| | | | | |
|---|---|---|---|---|
| Protein content (CNA), | 13.4 | 11.1 | 11.3 | 13.5 |
| % | | | | |
| Wet gluten, % | 33.1 | 29.0 | 32.9 | 34.6 |
| Minolta color - L* | 85.1 | 85.4 | 88.1 | 85.5 |
| a* | 0.70 | 0.78 | 0.42 | 0.75 |
| b* | 11.5 | 10.4 | 7.3 | 10.3 |
| Moisture, % | 12.4 | 12.5 | 12.0 | 12.7 |

TABLE 1c
Milling and Analytical Results (samples I through L)

| | | | | |
|---|---|---|---|---|
| Sample | I | J | K | L |

WHEAT (13.5% mb)

| | | | | |
|---|---|---|---|---|
| Moisture, % | 11.0 | 10.5 | 10.2 | 10.0 |
| Particle size index, % | 41 | 49 | 40 | 45 |

MILLING YIELD

| | | | | |
|---|---|---|---|---|
| Flour yield | 100.0 | 100.0 | 100.0 | 100.0 |
| (Whole grain flour), % | | | | |

GRANULATION

| | | | | |
|---|---|---|---|---|
| Over 60w | 11 | 12 | 11 | 9 |
| (340 μm), % | | | | |
| Over 6xx | 12 | 12 | 14 | 14 |
| (212 μm), % | | | | |
| Over 9xx | 23 | 20 | 23 | 23 |
| (150 μm), % | | | | |
| Thrus, % | 54 | 56 | 52 | 54 |
| Total recovery, % | 100 | 100 | 100 | 100 |

FLOUR (14.0% mb)

| | | | | |
|---|---|---|---|---|
| Protein content (CNA), % | 13.2 | 17.1 | 13.7 | 12.6 |
| Wet gluten, % | 34.3 | 49.2 | 33.9 | NES |
| Minolta color - L* | 85.1 | 85.6 | 84.7 | 84.1 |
| a* | 0.88 | 0.72 | 0.91 | 0.75 |
| b* | 10.8 | 9.9 | 10.5 | 11.0 |
| Moisture, % | 12.2 | 11.9 | 12.5 | 12.6 |

Similar kernel hardness, as evidenced by PSI results, was observed for all samples with the exception of sample G, which was observed to have kernel hardness characteristics similar to soft wheat, as shown in Tables 1a through 1c.

A wide range of protein content was observed among the samples. The lowest protein content was observed for samples F and G (approximately 11%), while the highest protein content was observed for sample J (17.1%). The majority of the samples tended to have protein contents around 13%. Wet gluten results tended to follow a similar trend to protein content.

Gluten strength characteristics were evaluated by GI, glutograph and gluten extensibility results. Results of these evaluations are shown in Tables 2a through c. Due to limited sample size gluten strength characteristics could not be evaluated for sample L, and limited analysis was completed on samples J and K. Seven of the ten samples, which included B, C, D, E, H, I and K, had GI values over 80%, indicating strong gluten properties. The lowest GI value was seen for sample G indicating weaker and more extensible gluten properties. Glutograph deflection time values were over 19 s for samples B, C, E, H, I and K indicating these samples have stronger gluten properties. Sample G showed the lowest glutograph deflection time indicating this sample had less resistance to deflection and therefore weaker gluten strength. Glutograph relaxation results relate to the elasticity of the gluten. Samples A, B, D, J, and K exhibited high relaxation values indicating greater gluten elasticity.

TABLE 2a
Gluten Strength Results (samples A through D)

| | | | | |
|---|---|---|---|---|
| Sample | A | B | C | D |

FLOUR (14.0% mb)

| | | | | |
|---|---|---|---|---|
| Gluten index, % | 74 | 92 | 92 | 83 |
| Glutograph - time, s | 15 | 26 | 19 | 16 |
| relaxation, BU | 212 | 198 | 158 | 196 |
| Gluten extensibility - peak force, g | 79 | 79 | 88 | 70 |
| extensibility, mm | 149 | 119 | 127 | 149 |

FARINOGRAM - 50 g

| | | | | |
|---|---|---|---|---|
| Absorption, % | 71.9 | 67.5 | 67.3 | 72.7 |
| Dough development time (DDT), min | 4.7 | 6.7 | 7.7 | 5.0 |
| Stability, min | 2.8 | 8.4 | 7.6 | 3.3 |
| Mixing tolerance index (MTI), BU | 66 | 22 | 34 | 49 |

TABLE 2b
Gluten Strength Results (samples E through H)

| | | | | |
|---|---|---|---|---|
| Sample | E | F | G | H |

FLOUR (14.0% mb)

| | | | | |
|---|---|---|---|---|
| Gluten index, % | 89 | 66 | 36 | 81 |
| Glutograph - time, s | 24 | 11 | 7 | 19 |
| relaxation, BU | 133 | 141 | 152 | 131 |
| Gluten extensibility - peak force, g | 68 | 61 | 67 | 91 |
| extensibility, mm | 126 | 170 | 174 | 146 |

FARINOGRAM - 50 g

| | | | | |
|---|---|---|---|---|
| Absorption, % | 68.4 | 64.5 | 62.5 | 67.5 |
| Dough development time (DDT), min | 5.0 | 3.7 | 3.2 | 5.5 |
| Stability, min | 12.4 | 2.3 | 2.5 | 4.7 |
| Mixing tolerance index (MTI), BU | 15 | 69 | 60 | 41 |

TABLE 2c
Gluten Strength Results (samples I through L)

| | | | | |
|---|---|---|---|---|
| Sample | I | J | K | L |

FLOUR (14.0% mb)

| | | | | |
|---|---|---|---|---|
| Gluten index, % | 91 | 64 | 84 | NES |
| Glutograph - time, s | 125 | 12 | 45 | NES |
| relaxation, BU | 2 | 279 | 185 | NES |
| Gluten extensibility - peak force, g | 80 | NES | NES | NES |
| extensibility, mm | 121 | NES | NES | NES |

FARINOGRAM - 50 g

| | | | | |
|---|---|---|---|---|
| Absorption, % | 71.0 | 76.4 | 68.5 | 70.4 |
| Dough development time (DDT), min | 6.8 | 7.0 | 7.7 | 4.2 |
| Stability, min | 13.8 | 8.3 | 9.6 | 3.2 |

High peak force values from the gluten extensibility results were observed for samples A, B, C, H and I. These samples all had peak force values greater than 79 g indicating an increased resistance to extension. Samples that showed the greatest extensibility, F and G, also tended to have lower GI and lower glutograph time values indicating weaker and more extensible gluten characteristics.

A range in farinograph absorption was observed among the samples, with sample G having the lowest absorption and sample J having the highest. Absorption generally follows a positive relationship with protein content and both of these samples had the lowest and highest protein contents and farinograph absorptions. Gluten strength properties are also evident from the farinograph results. Samples with strong gluten properties generally show higher stability and lower mixing tolerance index (MTI) values. Samples which showed high stabilities and low MTI values included B, C, E, I and K. These samples were also found to have strong gluten properties as evidenced by their high GI, glutograph time and gluten extensibility peak force values.

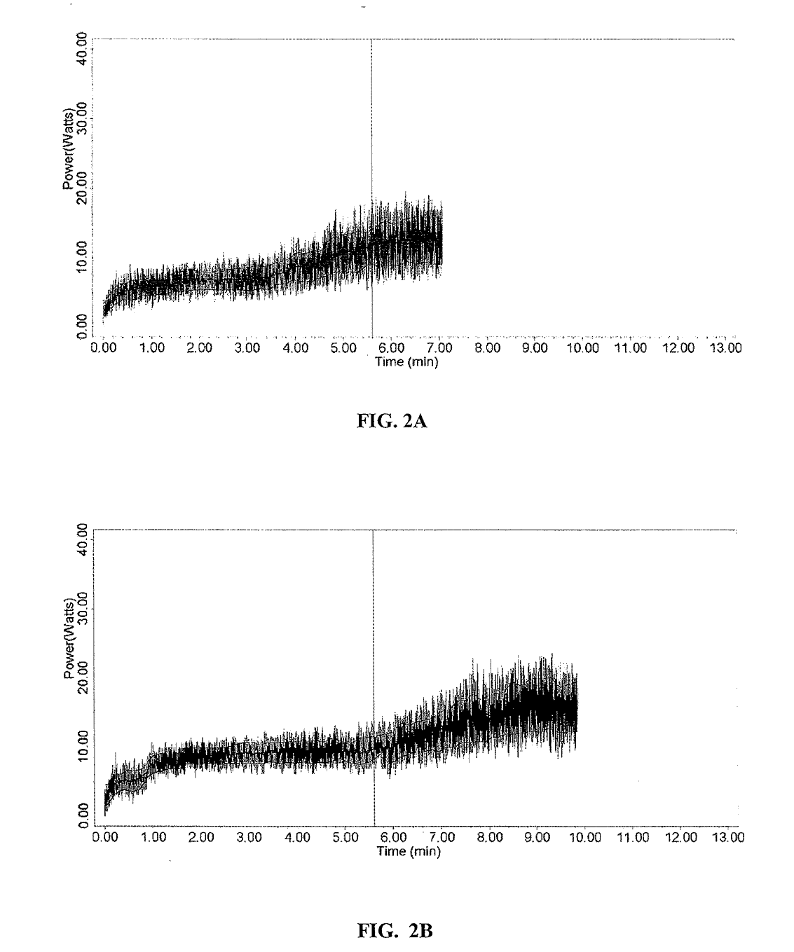

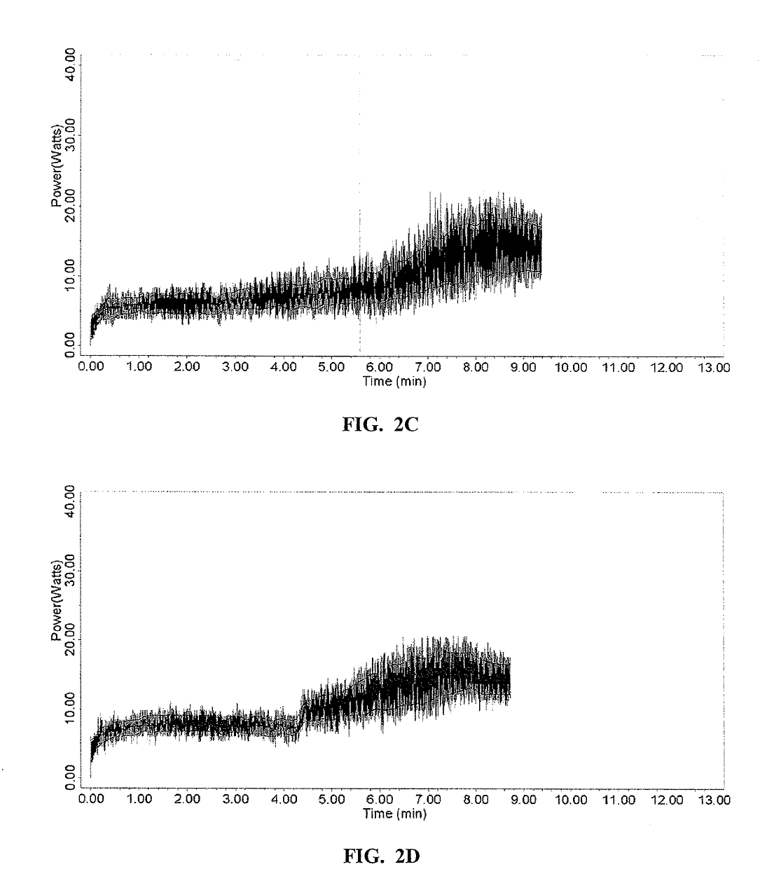

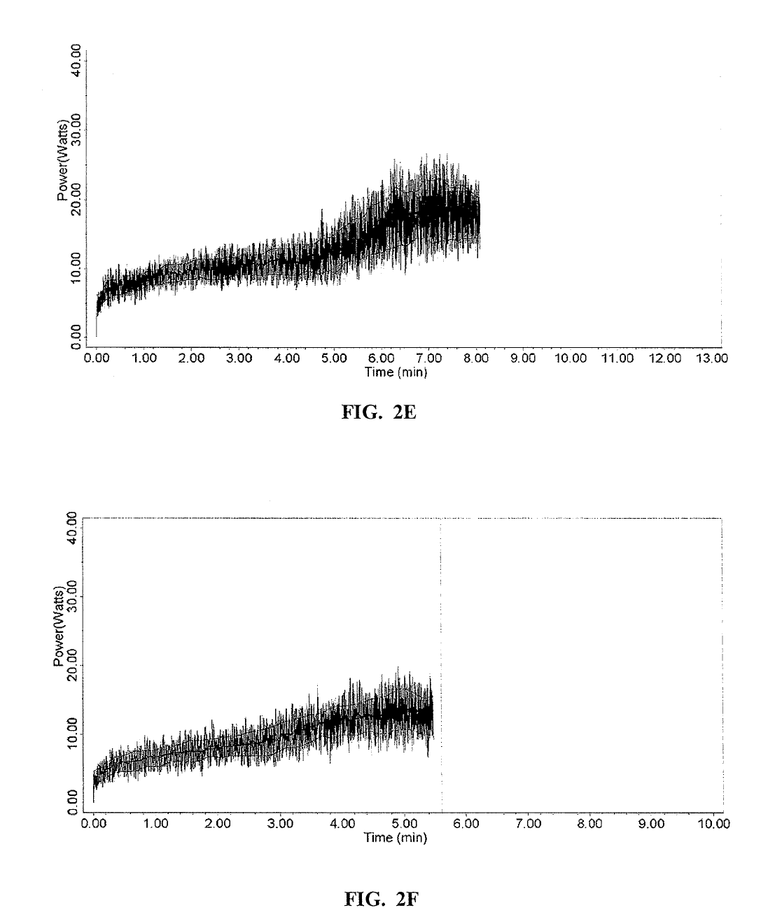

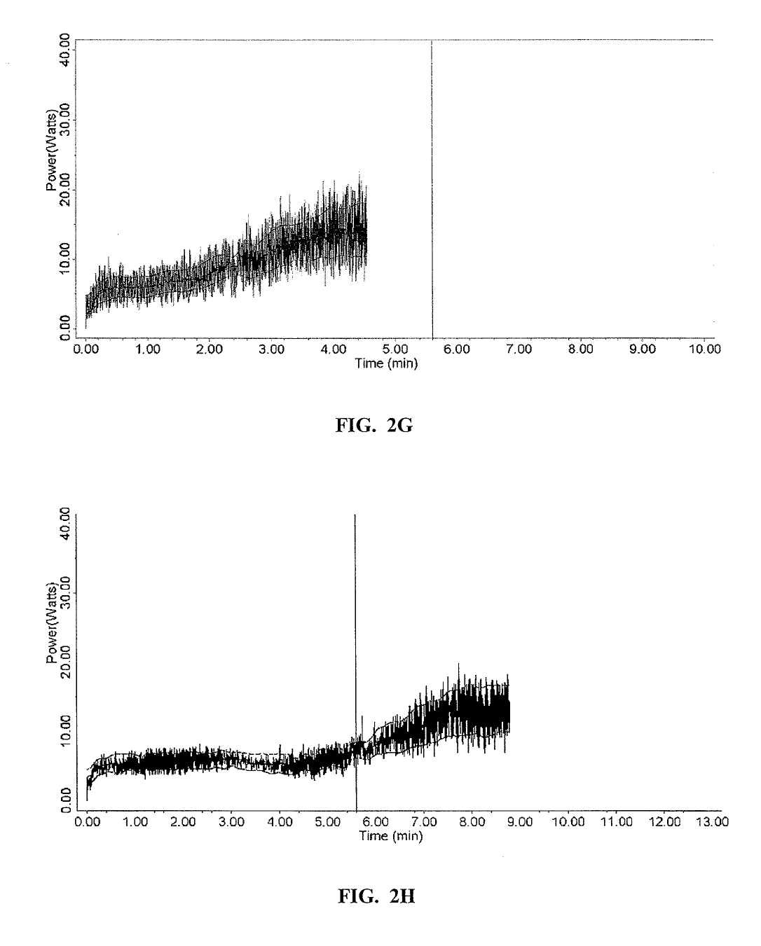

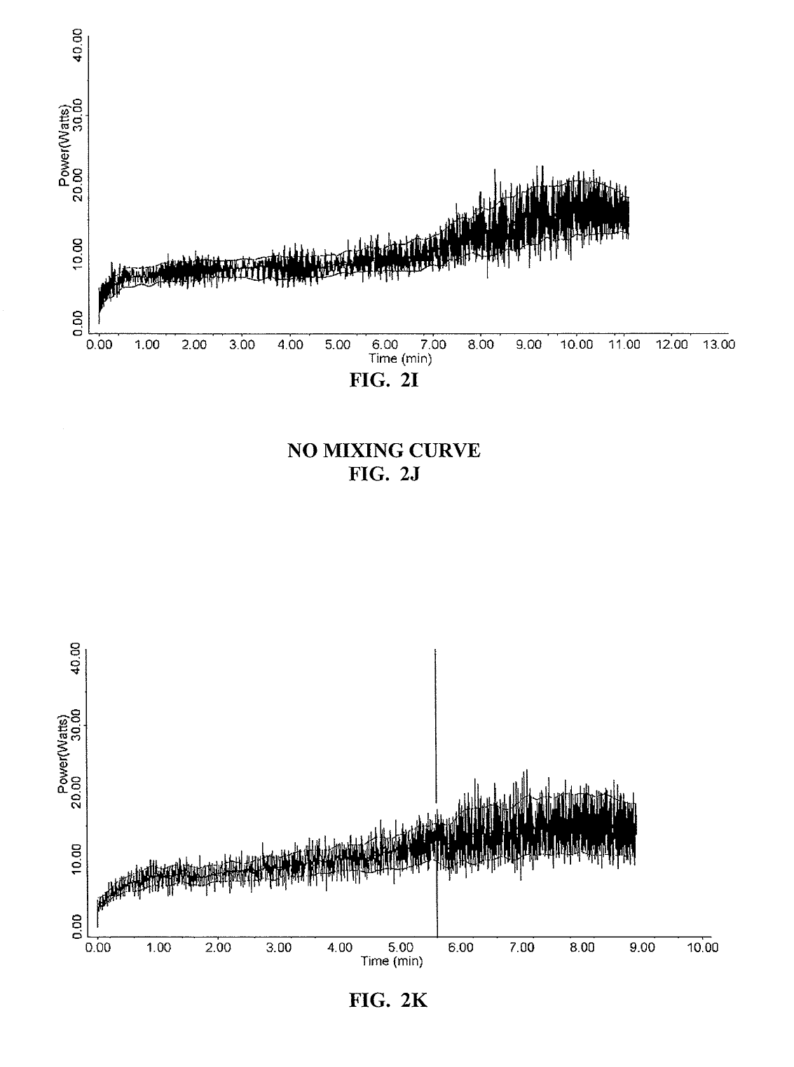

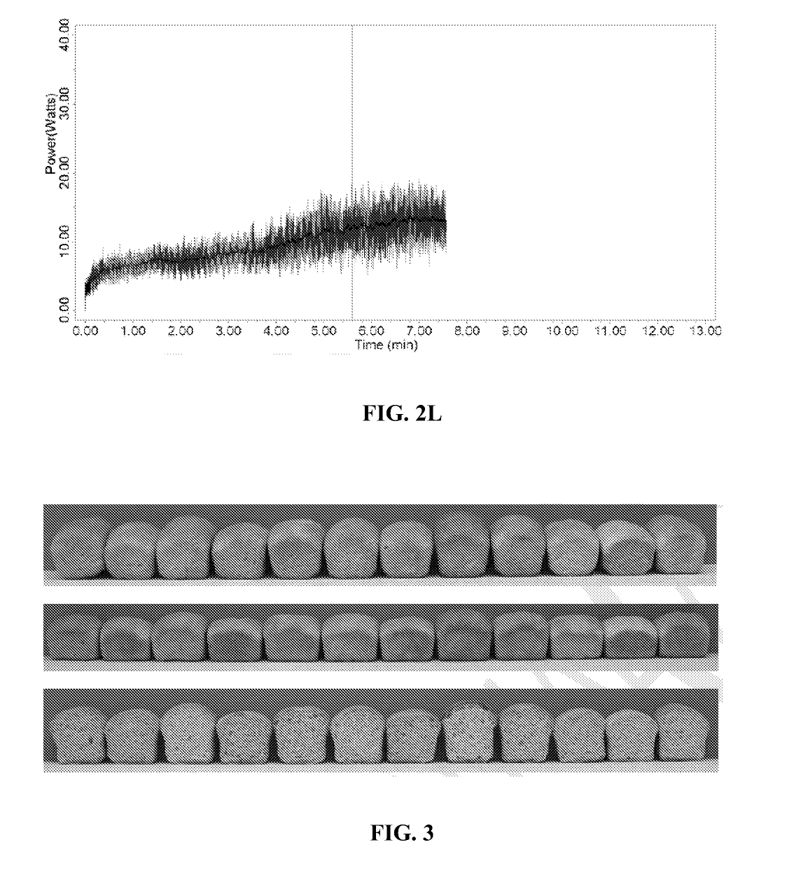

The 35 g micro test baking method provided useful data and exposed clear differences among the samples for volume, functionality, and whiteness (FIGS. 2 & 3; Tables 3a-3c). Mixing times of the samples ranged from 4.0-10.1 min, and were best explained by GI results as opposed to protein content (Tables 3a to 3c). This supports the use of GI as a useful indicator of dough strength, and supports the role of protein content for explaining baking absorption requirements. All samples processed well, but some of the samples, specifically A, D, E, I and J, showed stronger dough properties and improved handling. Dough handling indicated ease of processing the flour samples, but did not directly translate into improved loaf volume. For example, samples H and L exhibited weaker dough handling, average loaf volume (LV) and long mixing times, however, samples A and D exhibited strong dough handling, but below average LV and shorter mixing times. LV results ranged from 103 cc for sample A to 122 cc for sample J, however no apparent relationship between LV and other quality parameters was observed.

TABLE 3a
Test Baking Results (samples A through D)

| | | | | |
|---|---|---|---|---|
| Sample | A | B | C | D |

TEST BAKING (NO TIME DOUGH)

| | | | | |
|---|---|---|---|---|
| Baking absorption, % | 72.9 | 68.5 | 68.3 | 73.7 |
| Mixing time, min | 6.37 | 8.9 | 8.5 | 7.9 |
| Power, watt | 13.7 | 17.3 | 15.4 | 16.2 |
| Handling Comments | Strong | Slight Weak | Weak | Strong |
| Loaf volume | 103 | 116 | 105 | 104 |
| (TexVol), cc | | | | |
| Specific volume, | 2.6 | 3.0 | 2.7 | 2.7 |
| (TexVol), cc/g bread | | | | |
| External score | 20 | 15 | 16 | 18 |
| (out of 30) | | | | |
| Internal score | 43 | 42 | 42 | 41 |
| (out of 60) | | | | |
| Total Bread Score | 63 | 57 | 58 | 59 |
| (out of 90) | | | | |
| L*, (Minolta) | 66.6 | 66.2 | 65.9 | 65.8 |
| Whiteness order, | 11 | 8 | 12 | 9 |
| visual score | | | | |
| Cell contrastb | 0.66 | 0.71 | 0.66 | 0.71 |
| Cell diameter_{c}, | 2.16 | 1.85 | 1.94 | 1.79 |
| mm | | | | |
| Cell wall thickness_{d}, | 0.493 | 0.462 | 0.457 | 0.460 |
| mm | | | | |
| Slice area, mm2 | 1674 | 1633 | 1725 | 1521 |
| # of cells/areaₑ, | 1.83 | 1.54 | 1.63 | 1.59 |
| cells/mm₂ | | | | |

TABLE 3b
Test Baking Results (samples E through H)

| | | | | |
|---|---|---|---|---|
| Sample | E | F | G | H |

TEST BAKING (NO TIME DOUGH)

| | | | | |
|---|---|---|---|---|
| Baking absorption, % | 69.4 | 65.5 | 63.5 | 68.5 |
| Mixing time, min | 7.4 | 5.0 | 4.0 | 8.0 |
| Power, watt | 19.5 | 14.1 | 15.0 | 14.1 |
| Handling Comments | Strong | Good | Good | Weak |
| Loaf volume | 114 | 112 | 104 | 110 |
| (TexVol), cc | | | | |
| Specific volume, | 3.0 | 2.9 | 2.7 | 2.8 |
| (TexVol), cc/g bread | | | | |
| External score | 20 | 23 | 21 | 21 |
| (out of 30) | | | | |
| Internal score | 43 | 45 | 45 | 45 |
| (out of 60) | | | | |
| Total Bread Score | 63 | 68 | 66 | 66 |
| (out of 90) | | | | |
| L*, (Minolta) | 66.6 | 69.9 | 70.2 | 69.2 |
| Whiteness order, | 7 | 5 | 3 | 4 |
| visual score | | | | |
| Cell contrastb | 0.69 | 0.70 | 0.67 | 0.71 |
| Cell diameter_{c}, | 2.02 | 1.80 | 2.10 | 1.78 |
| mm | | | | |
| Cell wall thickness_{d}, | 0.469 | 0.451 | 0.477 | 0.452 |
| mm | | | | |
| Slice area, mm2 | 1725 | 1525 | 1837 | 1635 |
| # of cells/areaₑ, | 1.73 | 1.50 | 1.71 | 1.48 |
| cells/mm₂ | | | | |

TABLE 3c
Test Baking Results (samples I through L)

| | | | | |
|---|---|---|---|---|
| Sample | I | J | K | L |

TEST BAKING (NO TIME DOUGH)

| | | | | |
|---|---|---|---|---|
| Baking absorption, % | 72.0 | 77.4 | 69.5 | 71.4 |
| Mixing time, min | 10.1 | 5.1 | 8.1 | 6.8 |
| Power, watt | 17.5 | 23.3 | 16.3 | 13.9 |
| Handling Comments | Strong | Strong | Good | Weak |
| Loaf volume | 106 | 122 | 117 | 111 |
| (TexVol), cc | | | | |
| Specific volume, | 2.7 | 3.2 | 3.0 | 2.9 |
| (TexVol), cc/g bread | | | | |
| External score | 19 | 21 | 23 | 17 |
| (out of 30) | | | | |
| Internal score | 43 | 48 | 50 | 41 |
| (out of 60) | | | | |
| Total Bread Score | 62 | 69 | 73 | 58 |
| (out of 90) | | | | |
| L*, (Minolta) | 67.7 | 70.3 | 71.5 | 65.5 |
| Whiteness order, | 6 | 2 | 1 | 10 |
| visual score | | | | |
| Cell contrastb | 0.69 | 0.72 | 0.68 | 0.73 |
| Cell diameter_{c}, | 1.93 | 1.74 | 1.95 | 1.64 |
| mm | | | | |
| Cell wall thickness_{d}, | 0.476 | 0.452 | 0.475 | 0.441 |
| mm | | | | |
| Slice area, mm₂ | 1534 | 1490 | 1646 | 1715 |
| # of cells/areaₑ, | 1.72 | 1.50 | 1.67 | 1.33 |
| cells/mm₂ | | | | |

Gluten strength in the WG flours was assessed using several methods. Samples B, C, E and H all had strong gluten strength as evidenced by high values for GI, glutograph time, gluten extensibility peak force and farinograph stability and low MTI values. Sample K also showed high GI and stability and low MTI.

The results from sample G suggest that it is similar to soft wheat with soft kernel characteristics, low protein and weak gluten strength as measured by GI, glutograph and gluten extensibility results, and weak dough handling properties during baking. Overall, sample J exhibited the best baking quality with highest protein and wet gluten contents, short mixing time, strong dough handling properties, second whitest crumb and largest LV, and rounding out the top three were samples K then H.

Deposits of the Dow AgroSciences proprietary wheat cultivars AZCABR4421W, ARGIMI7232W, COI565W, AUBR31064W, AUBR31282W, CHBR1481W, and ARGBR5945W disclosed above and recited in the appended claims has been made with the American Type Culture Collection (ATCC), 10801 University Boulevard, Manassas, Va. 20110. The date of deposit was Nov. 11, 2010. The deposit of 2500 seeds for each variety were taken from the same deposit maintained by Dow AgroSciences since prior to the filing date of this application. All restrictions upon the deposit have been removed, and the deposit is intended to meet all of the requirements of 37 C.F.R. §1.801-1.809. The ATCC accession number for AZCABR4421W is PTA ______, for ARGIMI7232W is PTA ______, for COI565W is PTA ______, for AUBR31064W is PTA ______, for AUBR31282W is PTA ______, for CHBR1481W is PTA ______, and for ARGBR5945W is PTA ______. The deposit will be maintained in the depository for a period of 30 years, or 5 years after the last request, or for the effective life of the patent, whichever is longer, and will be replaced as necessary during that period.

While this invention has been described in certain example embodiments, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set forth in its entirety herein. The references discussed herein are provided solely for their disclosure prior to the filing date of the present application. Nothing herein is to be construed as an admission that the inventors are not entitled to antedate such disclosure by virtue of prior invention.

What is claimed is:

1. A wheat seed from a gluten quality wheat variety designated AZCABR4421W, ARGIMI7232W, COI565W, AUBR31064W, AUBR31282W, CHBR1481W, and ARGBR5945W.

2. A wheat plant produced from the seed of claim 1.

3. A tissue culture of cells from the plant of claim 2.

4. Tissue culture as recited in claim 3, comprising regenerable cells of a plant part selected from meristematic tissue, anthers, leaves, embryos, pollen and protoplasts therefrom.

5. A wheat plant regenerated from the regenerable cells of the tissue culture of claim 4.

6. Grain harvested from the plant of claim 2.

7. A grain product produced from grain harvested from at least one plant of claim 2.

8. The grain product of claim 7, wherein the grain product is a milled grain product.

9. The grain product of claim 7, wherein the grain product is a grain intermediate product.

10. The milled grain product of claim 8, wherein the grain product is an identity-preserved milled grain product.

11. The identity-preserved milled grain product of claim 10, wherein the identity-preserved milled grain product has the characteristic of high gluten quality.

12. The identity-preserved flour of claim 12, wherein the identity-preserved flour comprises more than 14% gluten proteins.

13. The identity-preserved flour of claim 12, wherein starch has not been removed from the identity-preserved flour.

14. Protoplasts produced from the tissue culture of claim 3.

15. A wheat plant regenerated from the tissue culture of claim 3, said plant having all the morphological and physiological characteristics of wheat variety selected from the group consisting of AZCABR4421W, ARGIMI7232W, COI565W, AUBR31064W, AUBR31282W, CHBR1481W, and ARGBR5945W, representative seed of said wheat varieties deposited under ATCC Accession Nos.

16. A method for producing an F1 wheat seed, comprising crossing the plant of claim 2 with a different wheat plant and harvesting the resulting F1 wheat seed.

17. A method of producing an herbicide, insect, or disease resistant wheat plant comprising transforming the wheat plant of claim 2 with a transgene that confers herbicide resistance or abiotic stress tolerance.

18. A baked product with a substantially uniform air cell structure, comprising wheat flour, with a reduced amount of added gluten in a concentration effective to make a baked product having a structure and height substantially the same as a corresponding product made with wheat flour, wherein the ratio of the gluten in the product is between about 0.13/lb to 0.16/lb of dough.

19. The baked product of claim 18, wherein the wheat flour is selected from the group consisting of wheat varieties designated AZCABR4421W, ARGIMI7232W, COI565W, AUBR31064W, AUBR31282W, CHBR1481W, and ARGBR5945W.

20. A dough capable of producing a baked product having a substantially uniform air cell structure, the dough comprising wheat flour, and a reduced amount of added gluten in a concentration effective to make a baked product having a structure and height substantially the same as a corresponding product made with wheat flour, wherein the ratio of the gluten in the baked product is between about 0.13/lb to 0.16/lb of dough.

21. The dough of claim 18, wherein the wheat flour is selected from the group consisting of wheat varieties designated AZCABR4421W, ARGIMI7232W, COI565W, AUBR31064W, AUBR31282W, CHBR1481W, and ARGBR5945W.

22. A flour produced from grain harvested from a high gluten wheat variety selected from the group consisting of AZCABR4421W, ARGIMI7232W, COI565W, AUBR31064W, AUBR31282W, CHBR1481W, and ARGBR5945W.

23. A food product made from the flour of claim 22.

24. The food product of claim 23 selected from the group consisting of bread, cake, or pasta.

25. A baked product having wheat flour a quality gluten wheat flour to allow for the addition from about 4 wt. % to 8 wt. % less gluten than that as defined as conforming to US Standard of Identity for whole wheat flour (21 CFR §137).

26. The baked product of claim 25 wherein the wheat flour is selected from the group consisting of AZCABR4421W, ARGIMI7232W, COI565W, AUBR31064W, AUBR31282W, CHBR1481W, and ARGBR5945W.
